(19) United States
(12) Patent Application Publication
(10) Pub. No.: US 2025/0381339 A1
(43) Pub. Date: Dec. 18, 2025

(54) VOLUME ESTIMATION FOR INFUSION PUMP

(71) Applicant: Takeda Pharmaceutical Company Limited, Osaka (JP)

(72) Inventors: John Kenneth Hainsworth, Cambridge (GB); James Kenneth Douie, Cambridge (GB); Rory James Cahill, Cambridge (GB); Matthew Ian Murchie, Histon (GB)

(73) Assignee: Takeda Pharmaceutical Company Limited, Osaka (JP)

(21) Appl. No.: 19/103,300

(22) PCT Filed: Aug. 17, 2023
(86) PCT No.: PCT/IB2023/000499

§ 371 Date: Feb. 12, 2025

Related U.S. Application Data
(60) Provisional application No. 63/399,114, filed on Aug. 18, 2022.

(51) Int. Cl. A61M 5/142; G01F 22/02

(52) U.S. Cl. CPC A61M 5/142; G01F 22/02; A61M 2205/3327; A61M 2205/3331

(57) ABSTRACT

A drug delivery system includes a single-use cassette driven by a reusable pump module. The cassette includes one or more drug delivery bags disposed in a pressure chamber. A pump pressurizes the pressure chamber through a pneumatic interface between the pump module and cassette to dispense the drug from the drug delivery bags into a valve block assembly and through an outlet port.

### CROSS-REFERENCE TO RELATED APPLICATIONS

This application claims the benefit of U.S. Provisional Application No. 63/399,114, filed Aug. 18, 2022, which is hereby incorporated by reference in its entirety.

### FIELD

Disclosed embodiments are related to drug delivery systems.

### BACKGROUND

Medicinal fluids are administered to patients through a variety of methods. These conventional methods typically include injection by a syringe, ingestion, or delivery by an infusion pump and needle. In the case of administration by an infusion pump, controlled volumes of medicinal fluids may be delivered to the patient at pre-programmed rates or automated intervals.

### SUMMARY

In some embodiments, a drug delivery system comprises a cassette comprising a pressure chamber, one or more drug delivery bags disposed within the pressure chamber, and an outlet port, wherein the one or more drug delivery bags are fluidly connected to the outlet port. The drug delivery system also comprises a pump module comprising a pump housing configured to removably couple to the cassette and a pump disposed within the pump housing. A pneumatic interface is formed between the pressure chamber and the pump when the cassette and the pump housing are in a coupled configuration, the pump configured to pump air into the pressure chamber through the pneumatic interface to pressurize the pressure chamber and cause a drug to flow from one of the one or more dug bags through the outlet port.

In some embodiments, a cassette comprises a housing, a pressure chamber disposed within the housing, the pressure chamber including an inlet port configured to selectively couple a pump to the pressure chamber to pressurize the pressure chamber, one or more drug delivery bags disposed within the pressure chamber, and a valve block assembly disposed within the housing configured to control drug flow from the one or more drug delivery bags.

In some embodiments, a valve block assembly for delivering a selected drug in a drug delivery system comprises a tubing system comprising a plurality of drug flow paths fluidly connecting one or more drug delivery bags to a single outlet port, and a valve switch configured to selectively block drug flow through at least one of the plurality of drug flow paths, the valve switch including a detent configured to maintain the valve switch in a desired blocking position.

In some embodiments, a cassette configured to be coupled to a pump module comprises one or more drug delivery bags disposed within the chamber, each of the one or more drug delivery bags comprising a septum, and a linkage movably coupled to the chamber, the linkage comprising a lever link and one or more spikes, whereupon coupling the chamber to the pump module in a first direction acts on the lever link to drive the one or more spikes in a second direction that is perpendicular to the first direction to pierce the septum each of the one or more drug delivery bags.

In some embodiments, a drug delivery system comprises a cassette comprising a first chamber, and a pump module configured to selectively couple to the cassette, the pump module comprising a second chamber that is pneumatically connected to the first chamber when the pump and the cassette are in a coupled configuration, wherein a ratio of a volume of the first chamber to a volume of the second chamber falls in a range between approximately 3:1 and 50:1.

In some embodiments, a method for estimating a volume of a drug in a collapsible drug delivery bag, the method comprises measuring a first air pressure in the first chamber when a collapsible drug-filled drug delivery bag is disposed in the first chamber. The method further comprises pneumatically connecting the first chamber to a second chamber and measuring a second air pressure in the first chamber. The method further comprises calculating a volume of air in the first chamber based on the first and second air pressure readings, an initial air pressure in the second chamber, and a known volume of the second chamber. The method further comprises subtracting the volume of air in the first chamber from a total volume of the first chamber.

In some embodiments, a method for detecting drug flow in a drug delivery system comprises estimating a volume of air in a first chamber, wherein a collapsible drug delivery bag is disposed in the first chamber. The method further comprises calculating a rate of pressure decay in the first chamber as a drug flows from the collapsible drug delivery bag. The method further comprises determining a calculated drug flow rate based on the rate of pressure decay and the estimated volume of air in the first chamber.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 illustrates a schematic of a drug delivery system, according to an embodiment;

FIG. 2 is a cassette, according to an embodiment;

FIG. 3 illustrates a pump module being coupled to an upper housing of a cassette, according to an embodiment;

FIG. 4 is an exploded view of a cassette, according to an embodiment;

FIG. 5 is a bottom housing of a cassette, according to an embodiment;

FIG. 6 is a pressure chamber and a valve block assembly, according to an embodiment;

FIG. 7 is an exploded view of a pressure chamber, according to an embodiment;

FIG. 8 is a top perspective view of a pressure chamber cap, according to an embodiment;

FIG. 9 is a drug delivery bag, according to an embodiment;

FIG. 10 is a pressure chamber cap with drug delivery bags, according to an embodiment;

FIG. 11 is a cross-sectional schematic of bag ports disposed in a pressure chamber cap, according to an embodiment;

FIG. 12 is a front perspective view of a valve block assembly, according to an embodiment;

FIG. 13 is a rear perspective view of a valve block assembly, according to an embodiment;

FIG. 14 is an exploded view of a valve block assembly, according to an embodiment;

FIG. 15 is a front view of a valve block chassis, according to an embodiment;

FIG. 16 is a rear view of a valve block chassis, according to an embodiment;

FIGS. 17A-17C illustrate a valve assembly and components thereof, according to an embodiment;

FIGS. 18A-18B are front and rear views of a valve switch, respectively, according to an embodiment;

FIG. 19 is a portion of the valve block assembly, according to an embodiment;

FIGS. 20A-20C illustrate positions of the valve switch, according to an embodiment;

FIG. 21 is an end of flow detector, according to an embodiment;

FIG. 22 is a schematic illustrating the operation of the end of flow detector;

FIG. 23 is a tubing system of the valve block assembly, according to an embodiment;

FIG. 24 is an exploded view of an outlet port, according to an embodiment;

FIG. 25 is a top, rear, perspective view of a valve block assembly, according to an embodiment;

FIG. 26 is a perspective view of a spike plate, according to an embodiment;

FIG. 27 is a perspective view of a pressure chamber and a valve block assembly, according to an embodiment;

FIG. 28 is a perspective view of a linkage mechanism, according to an embodiment;

FIG. 29 is a perspective view of a valve block assembly, according to an embodiment;

FIG. 30 is a perspective view of a cassette, according to an embodiment;

FIGS. 31A-31B illustrate a spike drive interface, according to an embodiment;

FIG. 32 is a lower housing of a cassette, according to an embodiment;

FIG. 33 is a schematic of bag ports disposed in a pressure chamber cap, according to an embodiment;

FIG. 34 is a pressure chamber cap with drug delivery bags, according to an embodiment;

FIGS. 35A-35B illustrate the operation of the linkage mechanism, according to an embodiment;

FIG. 36 illustrate a spike plate, according to an embodiment;

FIG. 37 illustrates a spike plate, according to an embodiment;

FIG. 38 illustrates spikes prior to piercing septums of bag ports, according to an embodiment;

FIG. 39 illustrates spikes after piercing septums of bag ports, according to an embodiment;

FIG. 40 is a schematic showing the operation of a drug delivery system, according to an embodiment;

FIG. 41 is a schematic of a drug delivery system, according to an embodiment;

FIG. 42 is a flow chart showing a method for drug volume estimation, according to an embodiment;

FIG. 43 is a flow chart showing a method for flow detection, according to an embodiment;

FIG. 44A is a perspective view of a plurality of drug delivery systems utilizing three different cassettes of different sizes;

FIG. 44B is a perspective view of the three different cassettes of different sizes shown in FIG. 44A;

FIG. 45A is a perspective view of the pressure chambers of the cassettes of FIG. 44B;

FIG. 45B is a perspective view of the drug delivery bag and tray arrangements of the pressure chambers of FIG. 45A;

FIG. 46A is a perspective view of the tray arrangement of the first cassette of FIG. 45B;

FIG. 46B is a perspective view of the tray arrangement of FIG. 46A with drug delivery bags;

FIG. 47 is a cross-sectional side view of a pressure chamber of the first cassette of FIG. 45B;

FIG. 48A is a perspective view of the tray arrangement of the second cassette of FIG. 45B;

FIG. 48B is a perspective view of the tray arrangement of FIG. 48A with drug delivery bags;

FIG. 49 is a cross-sectional side view of a pressure chamber of the second cassette of FIG. 45B;

FIG. 50A is a perspective view of the tray arrangement of the third cassette of FIG. 45B;

FIG. 50B is a perspective view of the tray arrangement of FIG. 48A with drug delivery bags;

FIG. 51A is a cross-sectional side view of a pressure chamber of the third cassette of FIG. 45B;

FIG. 51B is a perspective view of a pressure chamber body of the third cassette of FIG. 45B;

FIG. 52A is a perspective view of a valve block assembly according to an embodiment;

FIG. 52B is a perspective view of drug paths of the valve block assembly of FIG. 52A;

FIGS. 53A, 53B, 53C are top plan views of the valve block assembly of FIG. 52A, with a valve switch in three different positions;

FIG. 54 is a perspective view of a pinch valve of the valve block assembly of FIG. 52A;

FIG. 55A is a schematic of the pinch valve of FIG. 54 in a closed configuration;

FIG. 55B is a schematic of the pinch valve of FIG. 54 in an open configuration;

FIG. 56A is a perspective view of a pump module and a cassette separated from one another to show a sensing arrangement according to an embodiment;

FIG. 56B show the pump module and cassette of FIG. 56A in a coupled configuration, with portions of the pump module and cassette hidden or shown in phantom to illustrate the sensing arrangement;

FIG. 56C shows a portion of the cassette of FIG. 56B to illustrate a portion of the sensing arrangement;

FIGS. 57A, 57B, 57C are cross-sectional views of the pump module and cassette of FIG. 56A, with a valve switch shown in three different positions;

FIG. 58A is a bottom perspective view of the pump module of FIG. 56A, illustrating a portion of an end of flow sensing arrangements;

FIG. 58B is a perspective cutaway view of the pump module of FIG. 56A in a coupled configuration with the cassette of FIG. 56A, illustrating the end of flow sensing arrangement having a flow detector; and

FIG. 59 is a perspective cutaway view of the flow detector of FIG. 58B.

### DETAILED DESCRIPTION

Presently, preparation of drug therapies for home-based administration is a time-consuming process that involves many steps with the potential for error or contamination. For example, HyQvia therapy typically includes administering at least two drugs subcutaneously. A patient may first administer hyaluronidase (HY) to prepare the infusion site for receiving a large dose of the second drug, Immune Globin (IG). This process may include as many as 60 steps and many single-use packages to complete. A typical infusion cycle may last multiple hours.

In view of the above, the inventors have recognized and appreciated designs for a compact and portable drug delivery system that administers one or more drugs and that may do so in a simple method. In some embodiments, the drug delivery system includes a single-use cassette including one or more single-dose collapsible drug delivery bags and a reusable pump module that connects to the single use cassette module via a mechanical interface. The drug delivery system provides a single dose from the pneumatically compressed drug delivery bag within the single-use cassette. The pneumatic drive pressure is provided by the re-usable electromechanical pump module.

The system consists of a single-use cassette, containing a valve block assembly for drug selection and drug bags within a pressure chamber. The cassette attaches to a separate, re-usable pneumatic pump which provides pressurized air to the pressure chamber in order to dispense drug from the bags to a patient needle set. Subassemblies within the cassette enable patient selection of drug, end of flow detection and filling of drug bags by a pharmacist.

The drug delivery system maintains a compact design with few or no electronics in a cassette. In some embodiments, a patient may carry the drug delivery system with them during the infusion (e.g., via a strap, sling, backpack, handle, grip, harness, etc.).

In some embodiments, the drug delivery bags are contained within the single use cassette in a sterilized environment. The drug delivery system automatically administers the one or more drugs (e.g., HY and IG) through a single needle set without requiring the patient to handle the drug delivery bags. The system eliminates many steps in a typical HyQvia therapy. When the therapy is complete, the patient may discard or return the cassette to the manufacturer or to a pharmacy. The cassette may be sterilized and prepared with new drug delivery bags for future use.

With the infusion cycle typically lasting multiple hours, providing patients with an estimated time to completion is highly desirable, and ability to estimate the volume of drug remaining in a drug delivery bag is a prerequisite. However, due to constraints of current compact single use drug delivery system architecture, it is not practical to embed sensors or electronics in the single-use part of the system. The cassette may contain only a passive NFC label. It is only feasible to place sensing electronics in the pump module, and therefore any measurements of cassette state must be achieved only via the existing pneumatic interface between the Pump Module and Cassette Module.

In view of the above, the inventors have recognized and appreciate a method of indirectly determining the volume of the drug delivery bag via a single pneumatic interface between the pump module and cassette. The method may obviate the need for a sensor in the flow path and solve the problem of indirectly determining the rate of outflow from the drug delivery bag, thus allowing for a single pneumatic interface between single-use cassette and the reusable part of the system.

Although the disclosure describes a drug delivery system used with HyQvia therapy, it should be noted that any liquid based drugs that are capable of flowing out of a drug delivery bag when under pressure may be used with the system and methods described here, as the disclosure is not so limited. For example, any drug that may be stored in a drug delivery bag and squeezed out of the bag using pneumatic pressure may be used with the drug delivery system. Moreover, the drug delivery system is not limited to two-drug therapy with two drug delivery bags, but may administer single doses of drugs from one, two, or more drug delivery bags, depending on the prescribed therapy.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

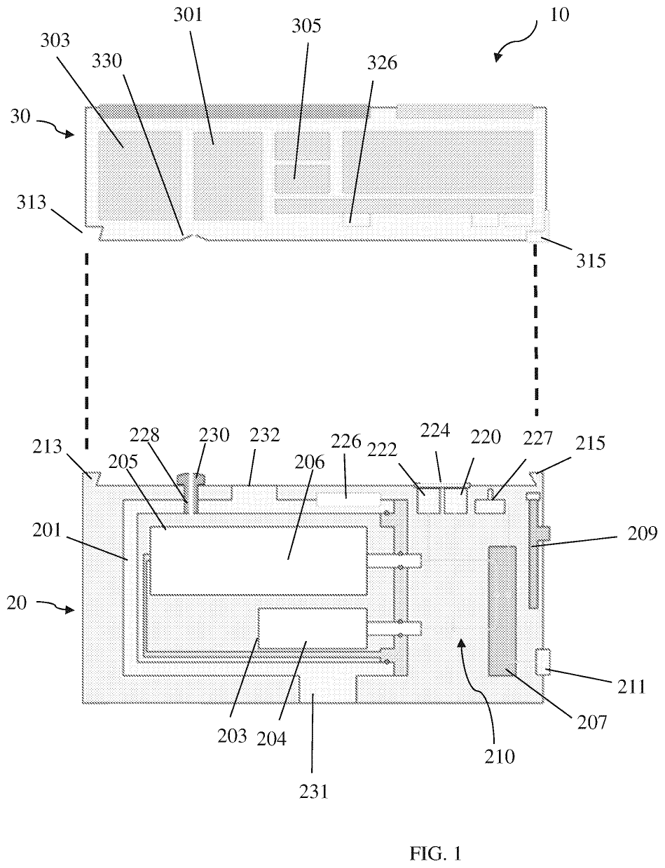

FIG. 1 shows a schematic of a drug delivery system 10 according to one embodiment. As shown in FIG. 1, the drug delivery system 10 includes a cassette 20 and a pump module 30. The cassette 20 includes a pressure chamber 201 which holds one or more collapsible drug delivery bags 203, 205. First drug delivery bag 203 may include a first drug 204, and second drug delivery bag 205 may include a second drug 206. In HyQvia therapy, for example, the first drug 204 may be HY and the second drug 206 may be IG. The drug delivery bags may be capable of holding large volumes of drugs (e.g., up to approximately 300 mL) to be administered to a patient subcutaneously. In some embodiments, the first drug delivery bag may have a capacity of up to approximately 30 mL and the second drug delivery bag may have a capacity of up to 300 mL. In some embodiments, the pressure chamber 201 may contain pressure which is pneumatically applied to the drug delivery bags.

Each of the drug delivery bags 203, 205 may be fluidly connected to a valve block assembly 207 that controls the flow path of each drug 204, 206 through and out of the drug delivery system 10. The valve block assembly includes a tubing system 210 through which the drugs may flow from the drug delivery bags to an outlet port 211. The pressure chamber 201 may be pressurized to squeeze and provide the motive force to the drug delivery bags and force either the first drug 204 or the second drug 206 drug out of the corresponding drug delivery bag into the tubing system 210 and through the outlet port 211. In this respect, the valve block coordinates which drug is able to flow out of its corresponding bag, as will be explained below. A needle system (not shown) may be connected to the outlet port 211 to administer the drug subcutaneously to a patient.

In some embodiments, the valve block assembly 207 ensures that no drugs or that only one of the drugs 204, 206 may be administered at a time. The valve block assembly 207 may include a switch 209 that allows a patient to select which drug to administer. The switch 209 may control mechanical elements that may be positioned to close off one or both of the drug flow paths from the drug delivery bags. For example, in HyQvia therapy, the switch may initially be in an “PAUSE” position in which the valve block assembly 207 prevents both drugs 204, 206 from flowing through the drug flow path through the outlet port 211. A patient may then set the switch 209 of valve block assembly 207 to a first position to allow the flow of HY 204 through the outlet port 211 of the valve block assembly 207. In some embodiments, the drug delivery system 10 may direct the pump module to adjust the air pressure in the pressure chamber 201 depending on which drug is flowing to control the flow rate of the drug. When the HY infusion is complete, the patient may then set the switch 209 to a second position to allow the flow of IG 206 through the outlet port 211. As such, a patient may use the drug delivery system 10 to administer one or more drugs, without the need to access the drugs inside the drug delivery system, through a single outlet port using only one needle system. Although described as the patient performing the steps during an infusion, it should be noted that a doctor, nurse, or other caretaker may perform any steps described herein to assist the patient in receiving the drug infusion from the drug delivery system 10. Accordingly, as used herein, “patient” may mean the patient themselves, or doctor, nurse, or other caretaker as appropriate for the context in which the term is used.

In some embodiments, the cassette 20 may be coupled to a pump module 30. When securely coupled together, the pump module 30 may provide pneumatic pressure to the pressure chamber 201 of the cassette 20 through pneumatic inlet port 228 to operate the cassette and administer a drug from a drug delivery bag. FIG. 1 shows the cassette 20 and pump module 30 in a non-coupled configuration. In some embodiments, to couple the pump module 30 to the cassette 20, a patient may engage a hinge catch 313 of pump module 30 with a hinge engagement 213 of cassette 20 at a first side and then secure the cassette and pump module together by engaging a clip 315 of pump module 30 with a clip engagement 215 of the cassette at a second side, as shown via the dashed lines in FIG. 1. The clip 315 may need to attain a certain position to indicate that the pump module has been securely attached to the cassette.

When securely engaged, a pneumatic seal 230 surrounding pneumatic inlet port 228 of the cassette 20 is arranged to mate with a sealing surface 330 of the pump module 30 to form an air-tight pneumatic interface between the cassette 20 and the pump module 30, allowing pump pressure to be communicated directly to the pressure chamber. The pump module 30 includes an air compressor pump 301 that may pump air into the pressure chamber 201 through the inlet port 228 to increase the air pressure within the pressure chamber 201. When the pressure chamber 201 is pressurized, the now pressurized air may apply pressure against an entire outer surface area of the drug delivery bags within the pressure chamber 201. The air pressure in the pressure chamber 201, when pressurized, may be greater than air pressure at an end of the drug flow path (i.e., an outlet port). Accordingly, when the valve block 207 opens a drug flow path between one of the drug delivery bags 203, 205 and the outlet port 211, the pressure in the pressure chamber 201 squeezes the drug delivery bag and causes the drug in the bag to flow out of the bag and through the tubing system 210 and the outlet port 211 of the valve block assembly 207 to a patient needle set attached to a patient. Although two drug delivery bags are shown, there may be embodiments in which more than one drug delivery bag may be used to deliver a first or second drug. For example, in some embodiments, there may be two or more drug delivery bags for a first drug (e.g., IG). There may be an open flow path between the two or more drug delivery bags and the corresponding outlet port. The disclosure is not limited to one drug delivery bag connected to an outlet port.

Because the air pressure presses on an entire surface area of the collapsible drug delivery bags, the drug delivery system can effectively empty a maximum volume of the drug from the bags. In some embodiments, the cassette 20 includes an end of flow detector 227. In some embodiments, the end of flow detector 227 may be a mechanical bellows-based, diaphragm-based, or any appropriate system that may change position when a drug delivery bag is empty of a drug. The position change may be detected by the pump module, as described in more detail below, to signal to the pump module 30 a drug deliver bag currently selected to administer a drug has been emptied. Accordingly, the end of flow detector 227 may indicate when a first drug delivery bag has been emptied to signal to the user to switch the valve block to open a subsequent drug path flow, and may indicate to the user the emptying of the last drug delivery bag for the given treatment sessions and that the drug therapy has completed.

The pump module 30 provides the necessary air pressure to drive the cassette 20 to administer the drugs to a patient. The pump module 30 includes most or all electronics required in the drug delivery system 10. In some embodiments, the pump module may include a battery 303 and one or more sensors 305 to measure the air pressure in the pressure chamber 201 of the cassette 20. In some embodiments, the pump module may include multiple pressure sensors designed to operate across different pressure ranges to improve accuracy of the pressure readings. The pump module may also include a pressor sensor to measure atmospheric pressure. As a result, no electronics, power sources, or sensors are required in the cassette 20 to control or monitor the drug delivery process. The cassette may include a passive NFC label in some embodiments. By reducing or eliminating electrical components in the cassette 20, the cassette 20 may have a compact, simplified design, that allows for easy portability and handling by a patient.

In some embodiments, the cassette 20 may be a single-use cassette that, after use, the patient may return to a provider. The cassette may be disassembled, and reusable parts may be sterilized and reused. In some embodiments, the cassette 20 may include windows 231, 232 that allow a user to view the contents of the drug delivery bags 203, 205. Although FIG. 1 shows two drug delivery bags 203, 205, this is merely representative and the cassette 20 may hold one or more drug delivery bags depending on the particular drug therapy. In some embodiments, the pump module 30 may be reusable and may be used repeatedly with different cassettes 20.

In some embodiments, the cassette 20 may include one or more fill ports 220, 222. The cassette 20 may be manufactured with empty drug delivery bags 203, 205 that may later be filled by a pharmacist or other provider. The pharmacist may receive an empty cassette 20 and fill the drug delivery bags with the required dose of drugs by accessing the fill ports 220, 222. As shown in FIG. 1, a first fill port 220 may be fluidly connected to the first drug delivery bag 203 and a second fill port 222 may be fluidly connected to the second drug delivery bag 205. As noted above, more than one drug delivery bag may be fluidly connected to the first fill port 220 and/or the second fill port 222. The fill ports 220, 222 may be needleless connectors (e.g., luer lock connectors) that may self-close when disconnected from a fill needle. When a pharmacist has finished filling the drug delivery bags, the pharmacist may attach a fill port cover 224 over the fill ports 220, 222 to prevent tampering with or contamination of the drug-filled drug delivery bags. The pharmacist may then ship the filled cassette to a patient.

In some embodiments, the cassette 20 may include an RFID label 226 to communicate with an RFID reader 326 on a pump module 30. The RFID label 226 may carry information such as the patient's identification, prescription, dry parameters, and other information. For example, a pharmacist may program the necessary parameters and information to the RFID label 226 when the subscription is filled. When the pump module 30 is coupled to the cassette 20, the pump module 30 may read the information on the RFID label 226 and automatically configure itself based on that information to administer the appropriate drug therapy. In some embodiments, the RFID label 226 may be able to detect temperature information for the cassette and transmit that information to the pump module 30. For example, the system may determine whether a temperature of a drug in a drug delivery bag located in the cassette is sufficiently similar to ambient temperature before beginning a drug therapy. As a result, no electrical connection between the cassette and pump module is required, providing a simple interface between the cassette and the pump module.

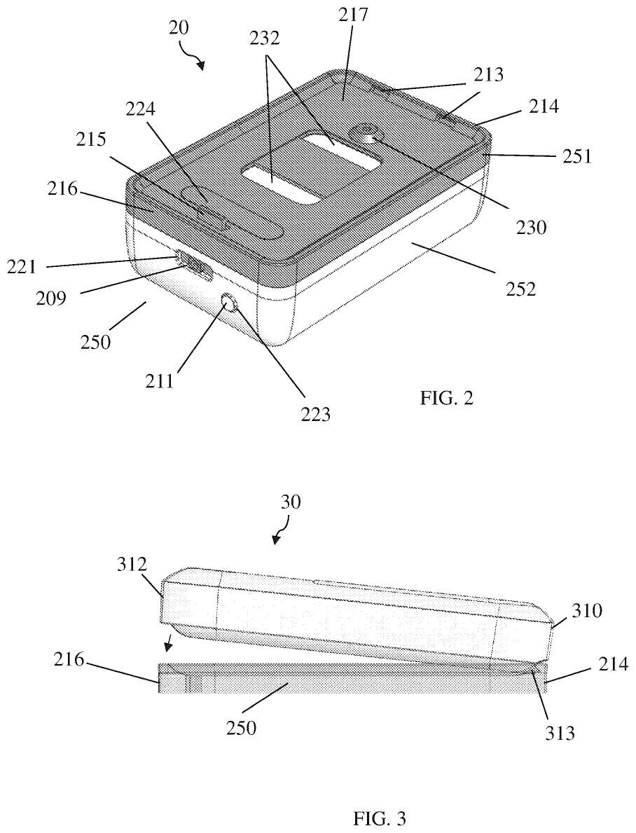

FIG. 2 shows a perspective top view of a cassette 20 according to an embodiment. The cassette 20 includes a housing 250 that holds the components of the cassette within an upper housing 251 and a lower housing 252. As described above, an upper surface 217 of the upper housing 251 may be shaped and sized to receive a pump module 30. The pump module 30 may hinge into the cassette 20 via a hinge interface and secured via a clip interface. In a non-limiting example, the upper surface 217 may include a hinge engagement 213 at a first side 214 of the cassette for receiving a hinge catch 313 of a pump module 30 (see FIG. 3). The upper surface 217 may also include a clip engagement 215 at a second, opposite side 216 to engage with a clip of the pump module to secure the pump module to the upper housing of the cassette.

FIG. 3 shows a side view of a pump module 30 being coupled to an upper housing 251 of a cassette, according to one embodiment. As shown in FIG. 3, a pump module 30 may be coupled to the upper housing 251 of the cassette 20 by first inserting a first side 310 of the pump module into the upper surface 217 at the first side 214 of the cassette such that the hinge catch 313 engages the hinge engagement 213 of the upper housing 251. A second side 312 of the pump module 30 may then be advanced toward the upper surface 217 at the second side 216 of the cassette until a clip 315 (see FIG. 1) of the pump module 30 engages with the clip engagement 215 of the upper housing to secure the pump module to the upper housing 251 in a coupled configuration.

In the coupled configuration, pneumatic seal 230 (FIG. 2) of upper housing 251 forms an air-tight seal with the pump module 30, forming a pneumatic interface between the pump module and cassette and connecting an air pump in the pump module to a pressure chamber in the cassette 20. It should be noted that any attachment mechanisms may be used to securely couple the pump module to the cassette, as the disclosure is not so limited.

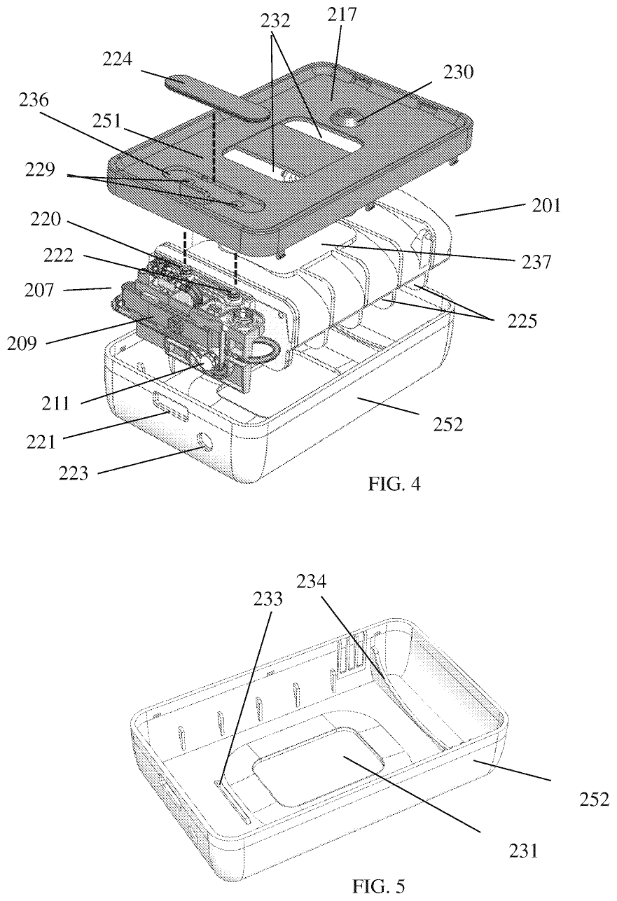

In some embodiments, as shown in FIG. 2, the upper surface 217 of the upper housing 251 may include one or more windows 232 to allow a patient to view contents of drug delivery bags within the housing 250. The lower housing 252 may also include one or more windows 231 on a lower surface of the housing 250 (see FIGS. 4-5).

In some embodiments, the housing 250 may include a first opening 221 providing access to a valve switch 209 and a second opening 223 through which the outlet port 211 may extend. As described above and further below, a patient may move the valve switch 209 to select a drug in one of the drug delivery bags in the cassette to flow out of outlet port 211.

FIG. 4 is an exploded view of a cassette 20, according to some embodiments. As shown in FIG. 4, an upper housing 251 and a lower housing 252 form a cavity that holds a pressure chamber 201 and a valve block assembly 207. FIG. 4 shows the upper housing 251 with a fill port cover 224 removed. The fill ports 220, 222 of the valve block assembly 207 may extend through openings 229 in the upper housing 251. As discussed with respect to FIG. 1, a specialist pharmacist may fill drug delivery bags disposed within the pressure chamber 201 via the fill ports 220, 222. When the drug delivery bags are filled with the prescribed dose of drugs, the pharmacist may attach the fill port cover 224 to preclude further access to the fill ports. The openings 229 may be formed in a cavity 236 in an upper surface 217 of the upper housing 251 having similar dimensions as the fill port cover 224 such that the fill port cover, when inserted into the cavity 236, may sit flush with the upper surface 217 (see FIG. 2). Although described as having an oval transverse cross-sectional shape, the pressure chamber may have any shape that houses the drug delivery bags efficiently while also withstanding internal pressure (e.g., rectangular, rectangular with rounded corners, similarly shaped to the drug delivery bags, etc.).

In some embodiments, the pressure chamber 201 may have an oval transverse cross-sectional shape along its longitudinal length. The oval shape may be similar to a shape of one or more drug delivery bags held within the chamber. One or more stiffening ribs 225 may extend radially from the pressure chamber in a transverse plane may at intervals along a longitudinal length of the pressure chamber 201. The stiffening ribs may help resist internal pressure when the pressure chamber is pressurized. The ribs 225 may form a generally rectangular shape that corresponds to inner surfaces of the upper and lower housings 251, 252. The ribs 225 may stabilize the generally oval-shaped pressure chamber within a rectangular shaped cavity formed by the upper and lower housings 251, 252.

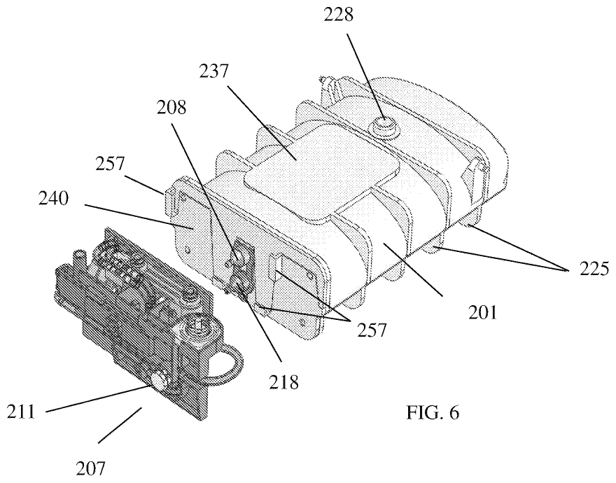

In some embodiments, as shown in FIG. 5, the lower housing 252 may include one or more pressure chamber retention ribs 233, 234 arranged on an inner bottom surface of the lower housing 252. A first retention rib 233 may abut against and retain a pressure chamber cap 240 (see FIG. 6) on an end of the pressure chamber 201 when the pressure chamber is pressurized. A second retention rib 234 may abut against a rib 225 of the pressure chamber. Second retention rib 234 have a curved upper surface that matches the oval shape of the pressure chamber such that a lower outer surface of the pressure chamber may rest flush against the retention rib 234. The retention ribs 233, 234 may help position and retain the pressure chamber 201 within the cassette housing 250.

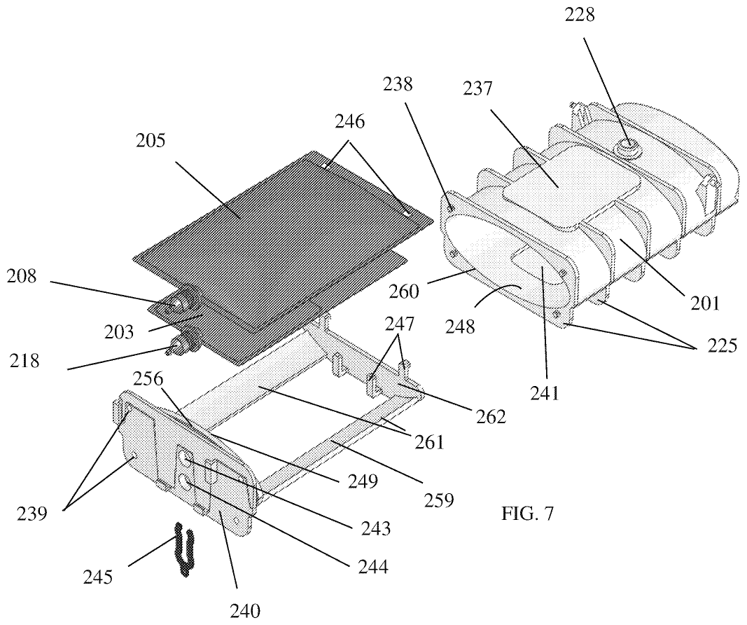

In some embodiments, the pressure chamber 201 includes a flattened, transparent surface 237 in an upper portion arranged under cassette windows 232 of the upper housing 251. As shown in FIG. 5, the lower housing 252 may include a window 231 that corresponds with a flattened, transparent surface 241 (see FIG. 7) arranged in a bottom portion of the pressure chamber 201. The flattened portions 237, 241 of the pressure chamber aid drug visibility through the cassette windows 231, 232 to allow a patient to view the quality of the drugs in the drug delivery bags disposed inside the pressure chamber 201.

FIG. 6 is a perspective view of a pressure chamber 201 and a valve block assembly 207 detached from the pressure chamber, according to some embodiments. As shown in FIG. 6, the pressure chamber 201 includes a pressure chamber cap 240 at one end of the pressure chamber. Bag ports 208 and 218 of drug delivery bags 203 and 205, respectively, extend through bag port openings in the pressure chamber cap 240. In some embodiments, the pressure chamber cap 240 includes one or more valve block grooves 257 that attach to and retain the valve block assembly 207 to the pressure chamber cap 240. When the valve block assembly 207 is attached to the pressure chamber cap 240, the bag ports 208, 218 are fluidly connected to the valve block assembly 207. Drugs may exit the drug delivery bags located in the pressurized pressure chamber 201 through the bag ports 208, 218 into the lower pressure flow path of the valve block assembly 207 and out through the outlet port 211.

FIG. 7 is an exploded view of a pressure chamber 201, according to some embodiments. As shown in FIG. 7, the pressure chamber cap 240 is rectangular in shape and matches the dimensions of a rectangularly-shaped rib 225 surrounding an opening 260 at an end of the pressure chamber 201. The rib 225 may include alignment pegs 238 extending from a front surface of the rib 225 that may be inserted into corresponding peg holes 239 of the pressure chamber cap 240 from a rear side of the cover when the cover is attached to the pressure chamber. In some embodiments, an oval-shaped seal 249 extends from the rear side of the pressure chamber cap 240 that is sized and shaped to fit snugly within an opening 260 of the pressure chamber. Sidewalls of the oval-shaped seal 249 form an air-tight seal with the inner surface 248 of the pressure chamber. In some embodiments, the oval-shaped seal 249 may include a groove 256 around a circumference in which a seal (e.g., o-ring) may be disposed to form an air-tight seal between the oval-shaped seal 249 and the inner surface 248. Additionally, the rear surface of the rectangular-shaped pressure chamber cap 240 may press against the front surface of the end rib 225 to provide a mechanical end-stop when the pressure chamber cap is fully inserted into the pressure chamber. The opening 260, being oval-shaped and the smallest cross-section of the pressure chamber, may provide a more robust seal with the pressure chamber cap 240.

In some embodiments, the pressure chamber cap 240 includes a bag chassis 259 that extends from the rear surface of the cover. The chassis 259 may include two-spaced apart legs 261 that extend between the cap 240 and an opposing vertical wall 262. The legs 261 and vertical wall 262 may be shaped to conform to the inner surface 248 of the pressure chamber 201. In some embodiments, the vertical wall 262 may be a half oval shape with the same curvature of the bottom inner surface 248 such that the vertical wall fits snugly against the inner surface 248. The legs 261 may extend from a portion of a perimeter of the oval seal 249 and be angled and curved to rest snugly against the bottom inner surface 248 of the pressure chamber 201.

In some embodiments, to attach the cap 240 to the pressure chamber 201, the chassis 259 may be slid into opening 260 along a longitudinal axis of the pressure chamber until the cap 240 contacts the rib 225 surrounding opening 260. The alignment pegs 238 pass through the alignment holes 239 in the cover to properly align the cap 240 and the chamber 201. The spaced-apart legs 261 are arranged on opposite sides of window 241 in the bottom portion of the pressure chamber to allow a patient to view the drug delivery bags 203, 205 in the pressure chamber.

In some embodiments, the pressure chamber cap 240 retains drug delivery bags 203, 205. The drug delivery bags may be arranged to lie flat along the chassis 259, with the bag ports 208, 218 at a first end of the bags extending through port openings, 243, 244, respectively, of the pressure chamber cap 240. A bag retention clip 245 may be positioned around the bag ports on a front surface of the cap to prevent the bags ports from being pulled out of the port openings and the drug delivery bags from shifting in the pressure chamber 201. The vertical wall 262 may include one or more bag hooks 247 that engage corresponding bag hook holes 246 in second ends of the drug delivery bags. In a non-limiting example, drug delivery bag 205 may include bag hook holes 246 spaced apart to correspond with upper, outer pair of hooks 247 on the vertical wall 262. Drug delivery bag 203 may include bag hook holes (not shown) spaced apart to correspond with lower, inner bag hooks 247 on the vertical wall. The port openings 243, 244 and bag hooks 247 help keep the drug delivery bags taut to maximize emptying of the drug during an infusion therapy.

In some embodiments, the drug delivery bags 203, 205 may accommodate a range of drug doses. For example, in HyQvia therapy, a smaller dose of HY is initially infused into a patient followed by a larger dose of IG. As shown in FIG. 7, drug delivery bag 203 containing a smaller dose of drug (e.g., HY) may have an inner volume that extends only partially along a length of the bag, with the remaining length of the bag having a flat, sealed portion to accommodate a length between the cap 240 and vertical wall 262. The smaller volume may be desirable to minimize any residual drug volume remaining in the drug delivery bag after therapy. Drug delivery bag 205, on the other hand, may contain a larger dose of drug (e.g., IG), and the inner volume may span an entire length of the bag to accommodate the larger dose. It should be noted that the drug delivery bags may have any relevant dimensions, shape, and arrangement within the pressure chamber, as the disclosure is not so limited. For example, the drug delivery bag 203 containing a lower volume of drug may not include a flat, sealed section, particularly when the drug being used is less expensive and the drug delivery bag can accommodate more residual volume of the drug. In addition, the drug delivery bags may not be arranged on top of one another, but may be arranged with at least one bag offset to one side of the chamber.

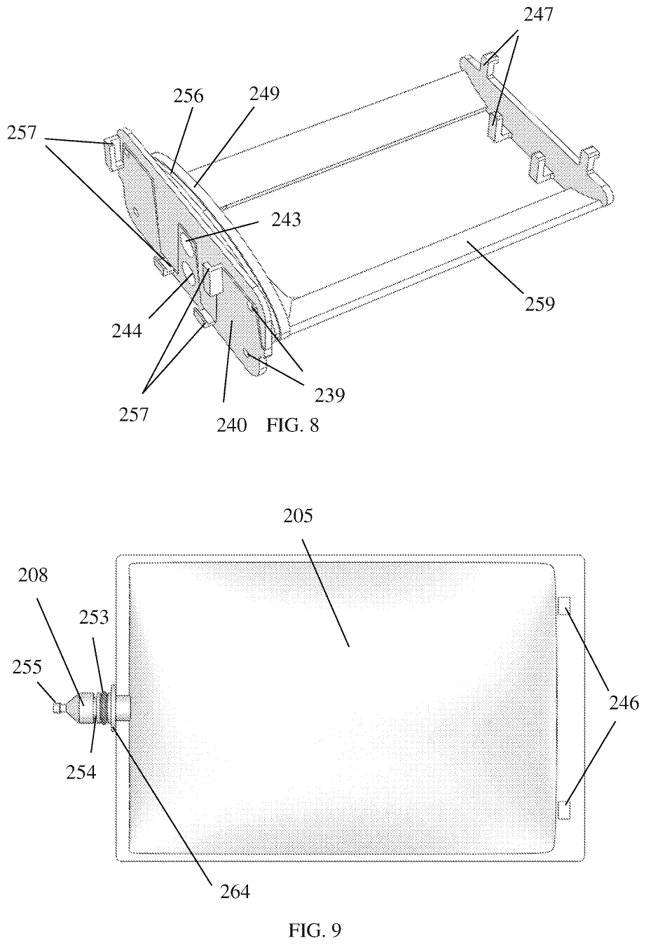

FIGS. 8 and 9 show larger views of a pressure chamber cap 240 and second drug delivery bag 205, respectively, according to some embodiments. As shown in FIG. 8, sidewalls of the oval-shaped seal 249 may include a groove 256 in which a pressure chamber O-ring may be disposed to provide an air-tight seal between the pressure chamber cap 240 and the inner surface 248 of the pressure chamber. Referring to FIG. 11, sidewalls 263 may include a groove 258 around an entire circumference to retain the pressure chamber O-ring.

As shown in FIG. 9, the bag port 208 may include a barbed tubing fitting 255 at a distal end for connecting the bag port 208 to a drug flow path of a valve block assembly 207. The drug port may allow the flow of drugs in and out of the drug delivery bags, either when a pharmacist fills the drug delivery bags with a drug or when the system is administering a drug during an infusion therapy. In some embodiments, bag port 208 may also include a retention clip groove 254 for receiving clip 245 and a bag O-ring 253 for forming a seal with the pressure chamber cap 240. It should be noted that although FIG. 9 described features of drug delivery bag 205, the features may apply to drug bag 203 and any other drug delivery bag used within the drug delivery system.

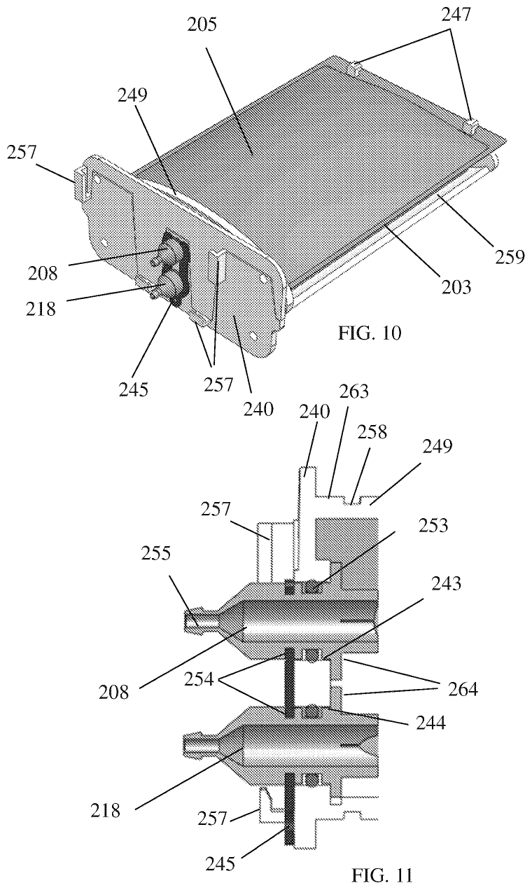

FIG. 10 shows the first and second drug delivery bags 203, 205 loaded into the pressure chamber cap 240. FIG. 11 shows an enlarged side schematic view of the bag ports 208, 218 extending through port openings 243, 244, respectively. As shown in FIG. 10, the bags are held in a flat position along chassis 259 and bag hooks 247 have engaged corresponding bag holes. Bag ports 208, 218 extend through the port openings in the pressure chamber cap 240 and are retained in the port openings with clip 245. As shown in FIG. 11, the bag ports 208, 218 include a disc 264 that extends radially from a proximal portion of the bag ports. The discs 264 abut a rear surface of the pressure chamber cap 240 to prevent the bag ports and drug delivery bags from moving distally. A clip 245 disposed within retention clip grooves 254 of the bag ports abuts a front surface of the pressure chamber cap 240 to prevent the bag ports and drug delivery bags from moving proximally. In some embodiments, the bag ports 208, 218 may include a groove in which a bag O-ring may be disposed to form a seal between the pressure chamber cap and the bag ports when the bag ports are positioned within port openings 243, 244. As a result, the pressure chamber cap 240 may simultaneously contain the pressure of the pressure chamber while allowing a liquid drug to pass through the bag ports out of the pressurized chamber to the valve block assembly.

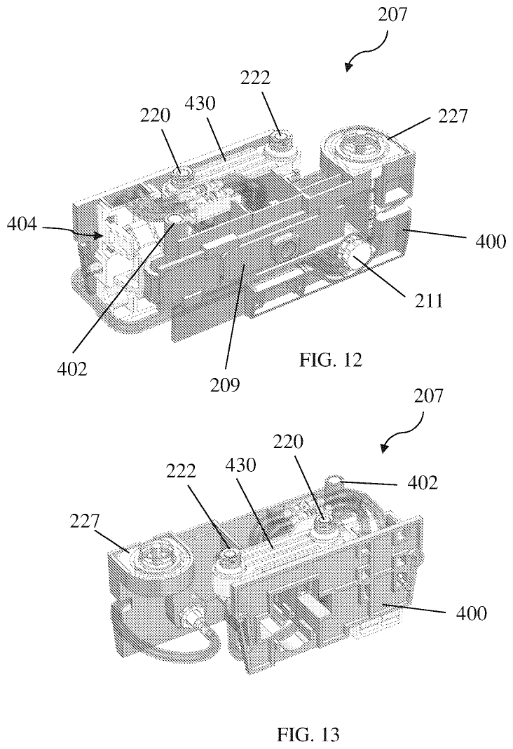

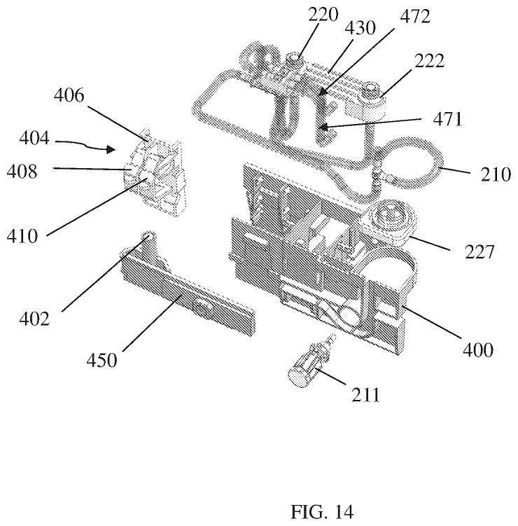

FIG. 12 shows a front perspective view of a valve block assembly 207, FIG. 13 shows a rear perspective view of the valve block assembly 207, and FIG. 14 shows a front, perspective, exploded view of the valve block assembly 207, according to some embodiments. As shown in FIGS. 12-14, in some embodiments, a valve block assembly 207 includes a valve block chassis 400 to which the components of the valve block assembly are mounted. The valve block assembly 207 may also include a mechanical switch 209, a valve subassembly 404, a filling port bracket 430, first and second fill ports 220, 222, a tubing system 210, an end of flow detector 227, and an outlet port 211.

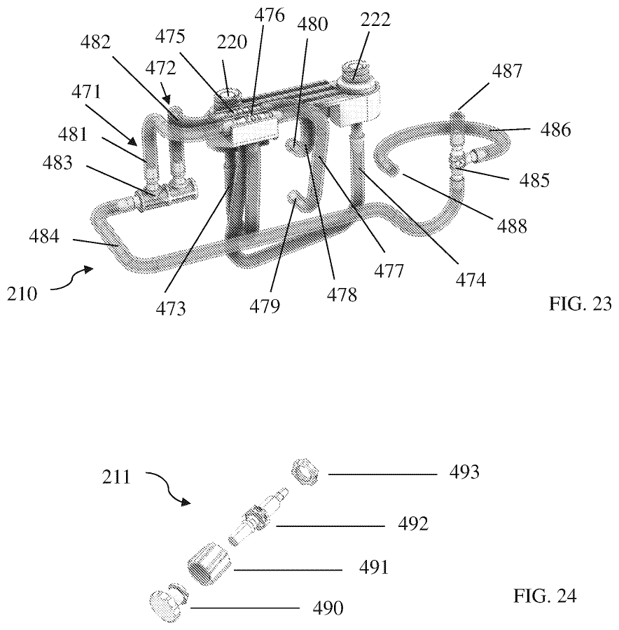

In some embodiments, the tubing system 210 includes a drug flow path for each drug in the drug delivery bags. As shown in the embodiment of FIG. 14, the tubing system includes two drug paths 471, 472 fluidly connected to the first and second drug delivery bags 203, 205, respectively. Each drug path 471, 472 includes a filling portion that connects the fill ports 220, 222 to drug delivery bags in the pressure chamber. The filling portion allows a pharmacist to fill the drug delivery bags with a prescribed dose of drugs via the fill ports. Each drug path 471, 472 also includes a flowing portion that connects the drug delivery bags to the outlet port 211. The tubing system 210 and drug flow paths 471, 472 are further described with respect to FIG. 23, below.

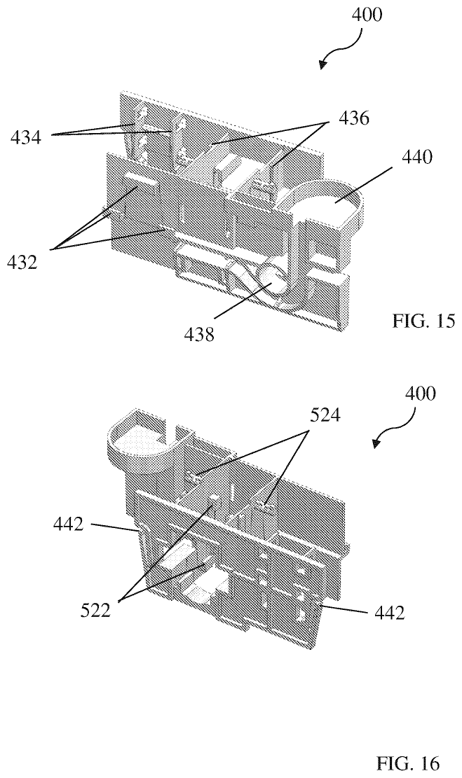

As shown in FIGS. 15 and 16, the valve block chassis includes switch bearing rails 432, valve mounting clips 434, filling port bracket mounting rails 436, an outlet port mounting hole 438, an end of flow detector pocket 440, and pressure chamber cap clips 442. The filling port bracket may be attached to the valve block chassis via the filling port bracket mounting rails 436. The outlet port 211 may extend through the outlet port mounting hole 438. The valve block chassis may attach to the pressure chamber cap 240 via the cap clips 442

As described above with respect to FIGS. 1 and 2, the valve block assembly includes a valve switch 209 that allows a user to select a drug to flow by sliding the switch to different positions. The switch may glide on the switch bearing rails 432 of the valve block chassis. As described in further detail below, the switch 209 may include a position indicator 402 (e.g., magnet) that communicates the switch state to control unit in the pump module 30. In some embodiments, the position indicator 402 may be a magnet and the pump module 30 may detect the position of the position indicator with a Hall-effect sensor. In some embodiments, the position of the valve switch may be detected using any appropriate approach (e.g., optical detection, sensors, etc.).

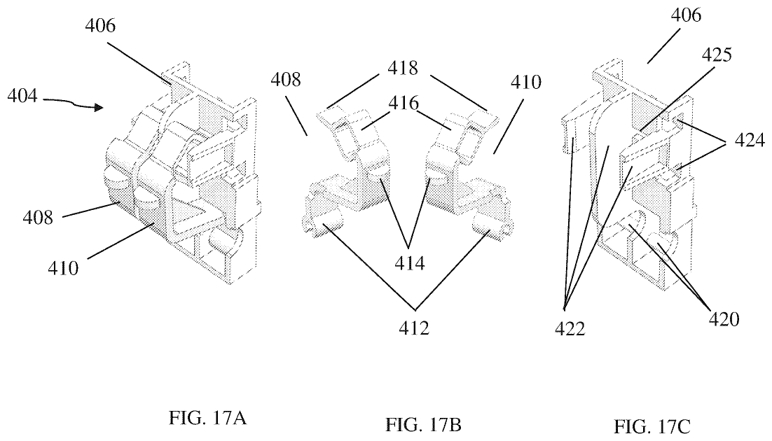

In some embodiments, the valve block assembly 207 includes a valve subassembly 404. The valve subassembly 404 controls the closing and opening of fluid drug paths in the drug delivery system. As shown in FIGS. 14 and 17A-17C, the valve subassembly may include a valve bracket 406 and first and second actuators 408, 410 disposed on the valve bracket 406. The valve assembly 404 may be attached to the valve block chassis via valve mounting clips 434.

In some embodiments, the actuators 408, 410 include clips 412, detents 414 flexures 416, and pinch tips 418 at a top end portion. In some embodiments, the valve bracket 406 includes actuator axles 420, guide fins 422, chassis clips 424, and slots through which pinch tips may extend 425. The clips 412 of actuators attach to the axles 420 of the valve bracket 406. The actuators are disposed between the guide fins 422. A top portion of the actuators include a flexure that may bend when pressed. The pinch tips 418 extend through slots 425 in the valve bracket. When assembled, the slots 425 are arranged adjacent to corresponding drug paths. As such, actuator 408 may be arranged in front of a first drug path and actuator 410 may be arranged in front of the second drug path. As described below, the switch may be moved to press against both or one of the actuators, causing the corresponding flexure to bend towards the drug path such that the pinch tip pinches the drug ling tubing and closes off the drug flow through that drug path.

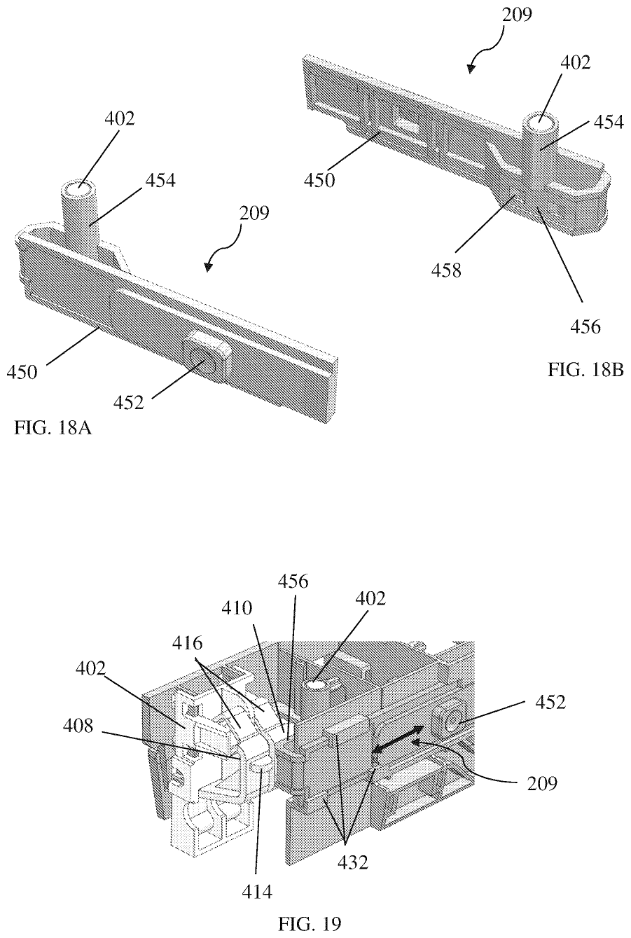

FIGS. 18A-18B show front and rear perspective views of a switch 209. As shown in FIGS. 18A-18B, the switch includes a slider body 450, a finger grip 452, a position indicator 402, a position indicator holder 454, an actuation surface 456, and detent recesses 458. A user may use the finger grip to slide the switch horizontally to select a drug flow state. The actuation surface is arranged to contact the actuators in the valve subassembly 404 in various positions. When the actuation surface 456 is slid in front of an actuator, the flexure of the actuator is displaced forward. The switch 209 may be moved such that the actuation surface contacts and deforms both or one actuator. Detent recesses 458 engage the detents 414 on actuators 408, 410.

When the switch is moved by a patient, it is important for the pump to recognize the position to which the switch is set to notify the patient of the system status and to verify that the cassette is correctly configured for a particular phase of the drug infusion therapy. Accordingly, if a patient sets the switch to the wrong position, the pump can send a notification to the patient to move the switch to the correct position. Accordingly, the system provides a non-contact method for sensing the switch position, as described below, which simplifies the mechanical interface between pump module 30 and cassette 20.

FIG. 19 shows an enlarged view of the switch 209 and the valve subassembly 404. The switch 209 may be slid horizontally in either direction to select a drug flow state by engaging with both or one of the actuators in the valve subassembly 404. Although described as a sliding valve switch, the disclosure is not so limited, and other appreciate types of switches may be used (e.g., rotational valve, three-way stopcock, etc.).

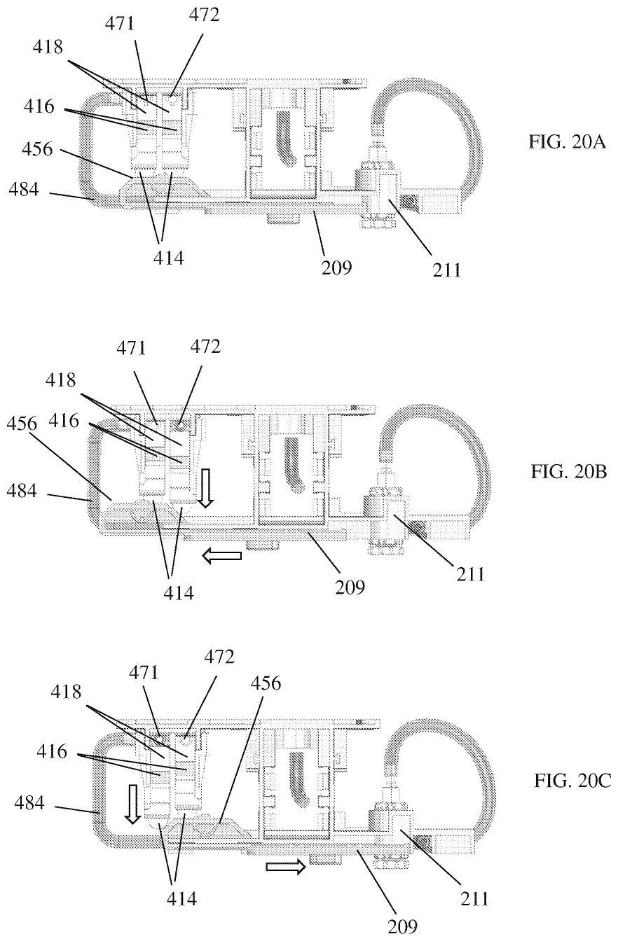

FIGS. 20A-20C illustrate three different positions of the switch 209, according to some embodiments. In some embodiments, the system may include a first drug path (e.g., for HY) and a second drug path 472 (e.g., containing IG). In a non-limiting example, in a system with two drug paths, the valve block assembly has three possible states: (1) both the first and second drug paths are closed; (2) the first path is open and the second path is closed; and (3) the first path is closed and the second path is open. The valve block assembly 207 ensures that the two drug paths may not be open at the same time.

The actuation surface of the switch pushes a valve actuator against the tubing of a fluid drug path to pinch it closed. To open the drug path, the switch can be moved to a position that allows the valve actuator to move away from the tubing, releasing the pinch. Each actuator incorporates a flexure which is intended to account for any manufacturing tolerances in the valving system, ensuring that the tubing is completely closed. One or more detents on the actuation surface of the switch holds the slider in each of the 3 positions shown in FIG. 20A-20C.

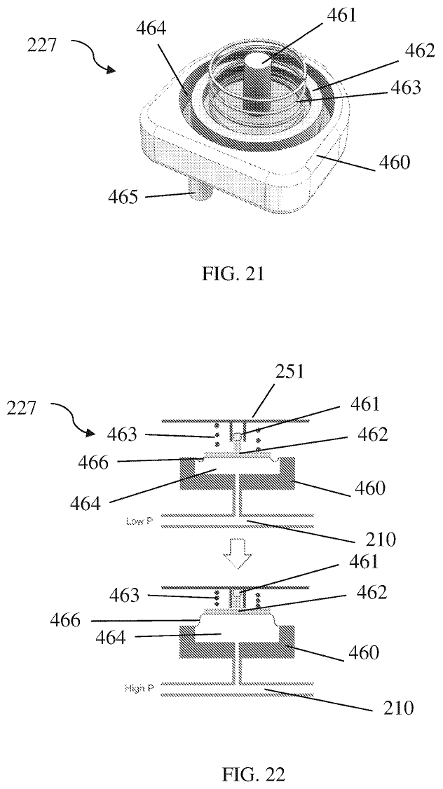

FIG. 21 shows an end of flow detector 227, according to some embodiments. The end of flow detector communicates to the pump module whether flow is occurring or has stopped. The end of flow detector 227 may be arranged in the end of flow detector pocket 440 of the valve block chassis. As shown in FIG. 21, the end of flow detector 227 may include a housing 460 and a magnet 461, a piston 462, and a spring 463. The magnet 461 may be disposed on the piston 462 which may linearly displace within the housing 460. The housing may include an inlet port 465 that connects to the tubing system 210 near an end of the drug path (see FIG. 23) A diaphragm seal 466 (see FIG. 22) between the piston 462 and the housing 460 allows the piston to linearly displace while maintaining a seal between the between the housing and the piston. The seal creates a pressure chamber within the cavity 464 which is fluidly connected to the drug path via inlet port 465.

FIG. 22 illustrates operation of an end of flow detector 227, according to some embodiments. The magnet 461 attached to the end of piston 462 changes position depending on whether there is drug flow in the tubing 210. Fluid pressure within the cavity may act on the diaphragm 466, which in turn presses against the piston 462. When there is drug flow, the cavity may be pressurized to the fluid pressure. A spring 463 may be sized such that it may be compressed at this fluid pressure during the drug flow. The spring compression may allow the piston 462 to be displaced upwards. When there is no drug flow from a drug delivery bag, there may no longer be fluid pressure exerted on the diaphragm and therefor the spring force may exceed the fluid pressure, causing the piston to retract. The magnet attached to the piston moves with the piston. A Hall-effect sensor in the pump module may detect the position of the magnet 461 to determine whether flow is occurring. FIG. 22 shows the end of flow detector 227 changing position from low fluid pressure to high fluid pressure. As noted above, in some embodiments, a position change of piston may be detected using an optical approach.

As such, the end of flow detector 227 may detect when a drug delivery bag is empty (i.e., when all contents have been dispensed). Although the pressure chamber 201 of the cassette is still pressurized at the end of an infusion, the end of flow detector 227 may be a mechanical diaphragm-based system that may change position when fluid is empty in bag.

FIG. 23 shows a perspective view of a tubing system 210 in the valve block assembly, according to some embodiments. As described above with respect to FIG. 14, the tubing system 210 may include first and second drug paths 471, 472. In some embodiments, the first and second drug paths 471, 472 include a first filling tube 473, 474 that extends between the filling ports 220, 222 and a first connector 475, 476. The filling ports 220, 222 allow the drug bags to be filled by a pharmacist prior to dispatch to a patient. In some embodiments, the filling ports may be a needleless injection site, a female luer lock to barb connector, or any known connector capable of receiving a liquid drug while maintaining a sterile, sealed environment.

The filling ports consist of off the shelf needle-free connectors, allowing connection of an external drug filling path to the cassette in order to fill the drug bags. The connectors can be sanitized before use and automatically seal upon disconnection.

In some embodiments, the drug paths 471, 472 include a second tube 477, 478 that attaches to the first connector 475, 476 at a first end and to a bag port of a drug delivery bag disposed in a pressure chamber at a second end 479, 480. For example, second end 479 of tube 477 may connect to the first drug delivery bag 203 and second end 480 of tube 478 may connect to the second drug delivery bag 205. When the cassette is being filled with first and second drugs through fillings ports 220, 222, the drugs flow through the first filling tubes 473, 474 and second tubes 477, 478 to the drug delivery bags. Accordingly, first filling tubes and second tubes may be referred to as a filling portion of the tubing system.

In some embodiments, third tubes 481, 482 may connect the first connectors 475, 476 to a second connector 483. A fourth infusion tube 484 extends between the second connector and a third connector 485. The third connector may include two outlet ports, one of which connects to an outlet tube 486 that connects to outlet port 211 and another which connects to an and of flow detector tube 487 that connects to an end of flow detector. The tubes and connectors that extend from the drug delivery bags to the outlet port may be referred to as an infusion portion of the tubing system. The second tubes 477, 478 and first connectors 475, 476 may be part of both the filling and infusion portions, and drugs may flow in either direction, either into or out of the drug delivery bags, depending on the state of the cassette.

When the drug delivery bags are filled through filling ports 220, 222, the switch 209 may be set in a first position in which the valve closes off drug flow through both third tubes 481, 482. As a result, when drugs are loaded into the cassette via the filling ports, the drugs will flow through first fillings tubes 473, 474 to first connectors 475, 476, and then flow through second tubes 477, 478 rather than through third tubes 481, 482, which are blocked by the valve. The drugs will flow into the drug delivery bags in the pressure chamber, which has not yet been pressurized by a pump module. During the infusion process, however, the pressure chamber may be pressurized and the valve may open drug flow through one of the third tubes 481, 482. Due to higher pressure in the pressure chamber and lower pressure at the outlet port 211, drugs will flow from the drug delivery bags and through the infusion portion of the tubing system to the outlet port 211.

FIG. 24 shows an exploded perspective view of an outlet port 211, according to some embodiments. As shown in FIG. 24, the outlet port may include an end cap 490, a luer lock 491, a panel mount connector 492, and a nut 493. In some embodiments, the outlet port may connect to a needle set to administer the drug to a patient. The drug delivery system provides a system that is capable of administering more than one drug through a single needle set. In some embodiments, the outlet port provide may be a one-way port to prevent drugs from reentering the cassette.

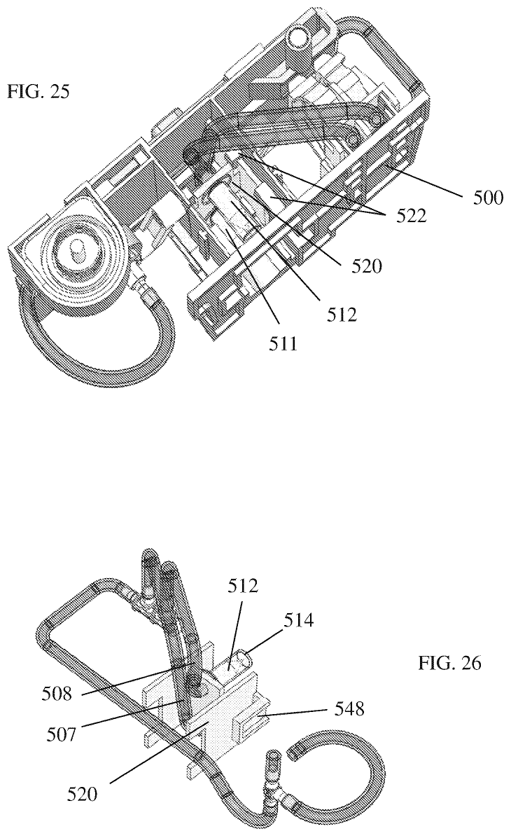

FIGS. 25-39 illustrate a cassette 50 and components thereof, according to another embodiment. Cassette 50 is similar to and includes many of the same features of cassette 20 described above with respect to FIGS. 2-24. Similar features will not be described with respect to cassette 50. While cassette 20 is described as being a pharmacy-filled cassette, cassette 50 may be designed to be factory assembled with pre-filled drug delivery bags in an automated factory-based filling line. Due to drug stability constraints in storage, the one or more drugs may only be allowed to contact the valve block assembly drug flow path shortly before an infusion therapy commences. In this embodiment, the bag ports of drug delivery bags are sealed when the cassette is assembled. Only when a patient couples a pump module 30 onto the cassette 50, the drug delivery bags may be unsealed to the valve block assembly.

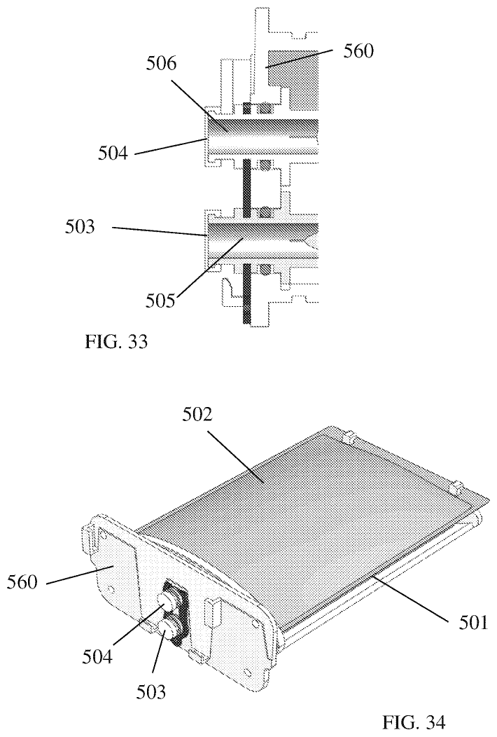

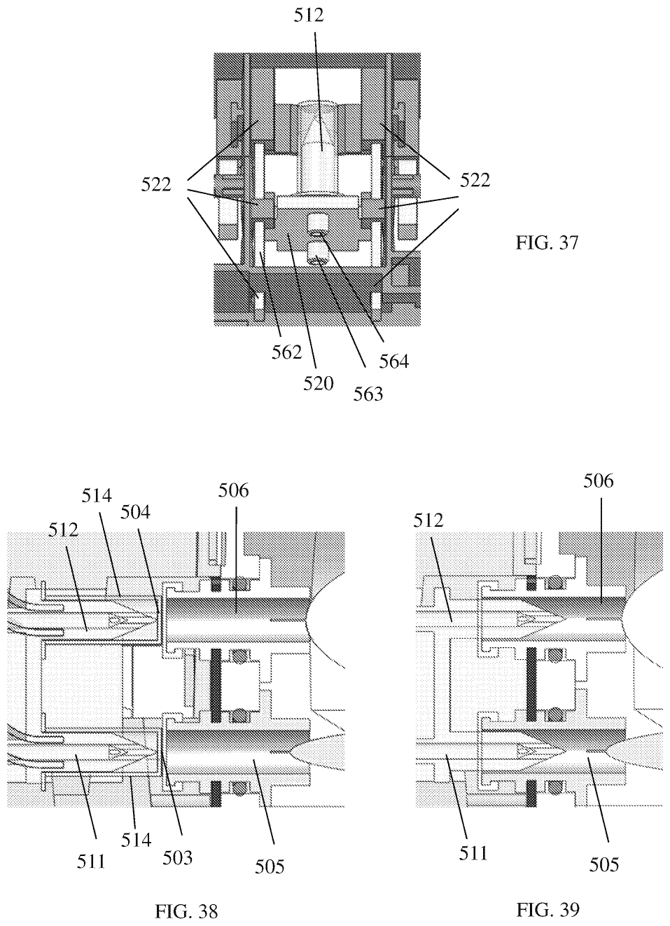

In some embodiments, the drug delivery bags 501, 502 are sealed with a septum 503, 504 at the end of bag ports 505, 506 (see FIGS. 33-34). The septum 503, 504 may be pierced by spikes within a valve block assembly 510 when the pump module is coupled to the cassette 50. FIGS. 25-26 show spikes 511, 512 arranged in the valve block assembly 510. As shown in FIGS. 25-26, the first drug path 507 and second drug path 508 of the valve block assembly 510 may be connected to spikes 511, 512 rather than directly to the drug delivery bags. The spikes 511, 512 may be covered with spike sheaths 514 to maintain sterility up until the point of spiking. In some embodiments, spikes 511, 512 are disposed on a spike plate 520. The spike plate 520 may include walls or rails for engaging spike plate bearings 522 of the valve block chassis 500 (see also FIGS. 16 and 37).

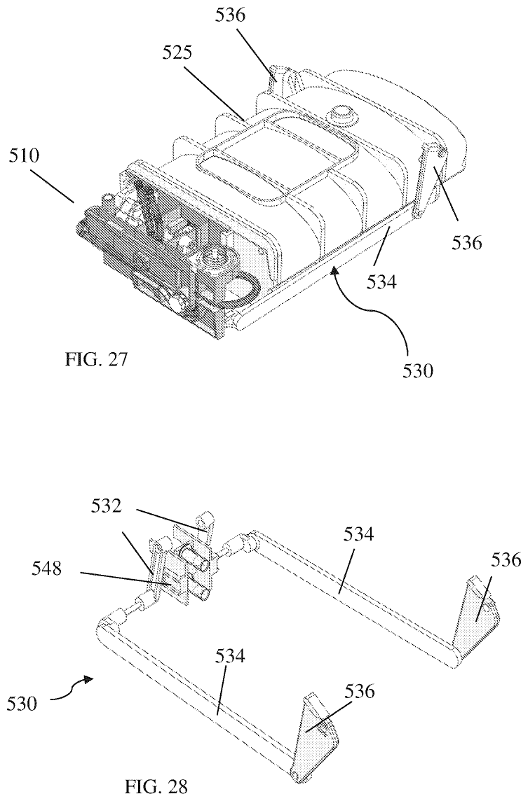

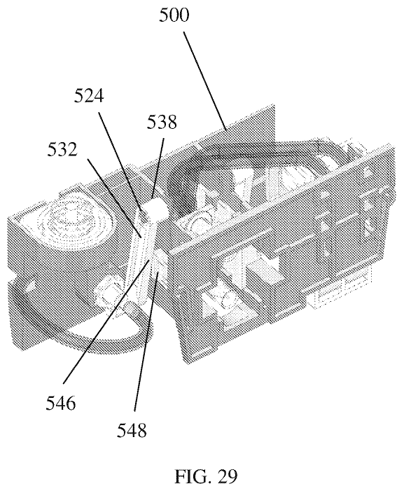

As shown in FIGS. 27-28, a linkage mechanism 530 may be mounted to a pressure chamber 525 and valve block chassis 500 to drive the spikes 511, 512 into the drug delivery bag septums upon coupling a pump module to the cassette 50. In some embodiments, the linkage mechanism 530 may be a 4-bar linkage that includes a pair of spike links 532, a pair of long links 534 that extend along a length of the pressure chamber, and a pair of lever links 536. In some embodiments, the pair of lever links 536 may be pinned at the pressure chamber and the pair of spike links 532 may be pinned at the valve block chassis 500. As shown in FIG. 29, ends 538 of spike links 532 may include through holes that are rotatably coupled to spike link axles 524 of the valve block chassis 500 (see also FIG. 16).

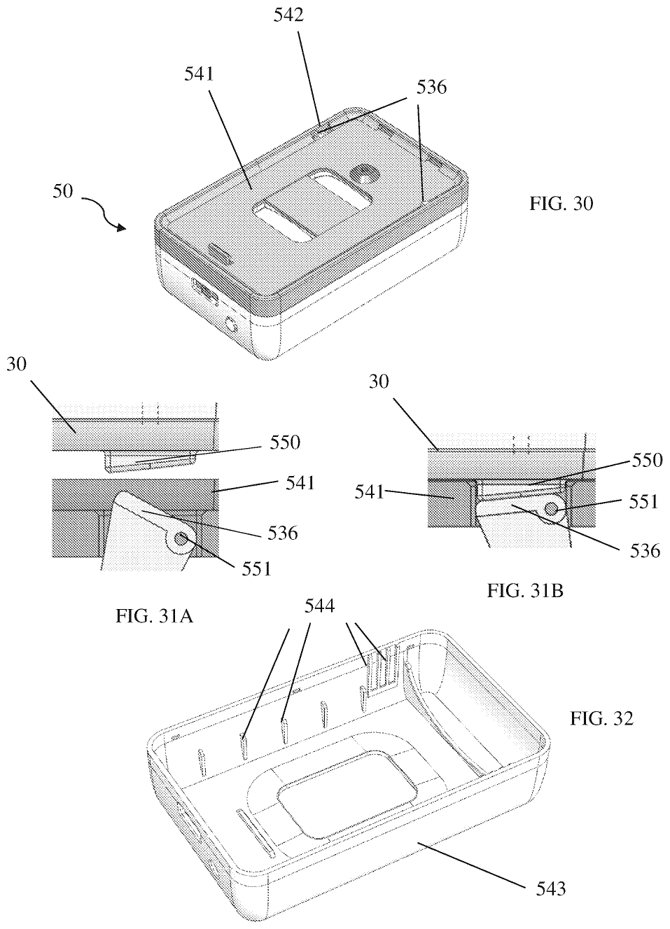

In some embodiments, the pair of lever links 536 protrude from openings 542 in upper housing 541 of cassette 50, as shown in FIG. 30. FIGS. 31A and 31B show the pump module being coupled to an upper housing of the cassette 50. In some embodiments, the pump module 30 may include a spike drive interface 550 arranged on a bottom surface of the pump module to extend into openings 542 of the upper housing 541 when the pump module is coupled to the cassette 50. In some embodiments, the linkage mechanism 530 converts downwards motion of the pump spike drive interface 550 to a horizontal spike displacement of the spike plate 520 to drive the spikes 511, 512 into septums of the drug delivery bags.

As shown in FIGS. 31A-31B, the pair of lever links 536 may be rotatably attached to pin 551 of the pressure chamber. Downwards motion of the spike drive interface 550 causes the level links 536 to be pressed down and rotated in a counter-clockwise direction about pin 551. Rotation of the lever links 536 causes the long links to displace in a horizontal direction. In some embodiments, as shown in FIG. 32, a lower housing 543 of cassette 50 includes one or more linkage running ribs 544 to aid the alignment of the long links 534. Horizontal displacement of the long links causes the spike links 532 to rotate about the spike link axles 524 in a direction toward the pressure chamber 525.

FIGS. 33 and 34 illustrate the drug delivery bags 501, 502 disposed in the pressure chamber cap 560. The bag ports 505, 506 extend through openings in the pressure chamber cap, similar to the embodiments described above with respect to FIGS. 10-11. The bag ports are sealed with septums 503, 504 to prevent drugs in the drug delivery bags from passing into a valve block assembly until the pump module is coupled to the cassette. Once the septums have been pierced, the drug flow operates in a similar manner as described above. The pressure chamber cap 560 allows for drugs to pass through the bag ports of the drug delivery bags while maintaining a sealed interface to maintain the pressure in the pressure chamber.

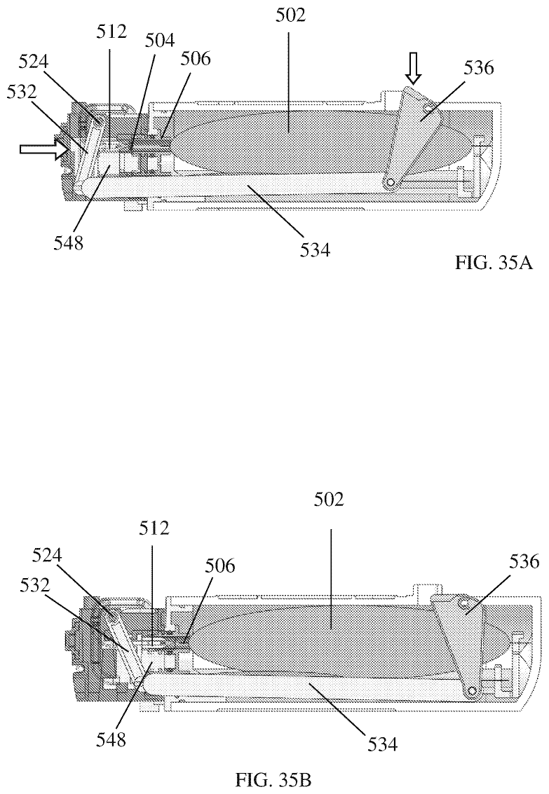

FIGS. 35A-35B illustrate the process of piercing the septums 503, 504 with spikes 511, 512. FIG. 35A shows the linkage mechanism prior to pump module coupling and FIG. 35B shows the linkage mechanism after pump module coupling. The downward force of the pump module, as described above, is converted into horizontal displacement of the spike plate 520. As shown in FIG. 35B, the spike links 532 have been rotated counterclockwise about spike link axle 524, causing the link to move towards the pressure chamber. This movement causes the spike plate 520 to displace horizontally towards the pressure chamber, driving the spikes 511, 512 towards bag ports 505, 506 to pierce septums 503, 504. Piercing the septums opens the flow path to the valve block assembly 510.

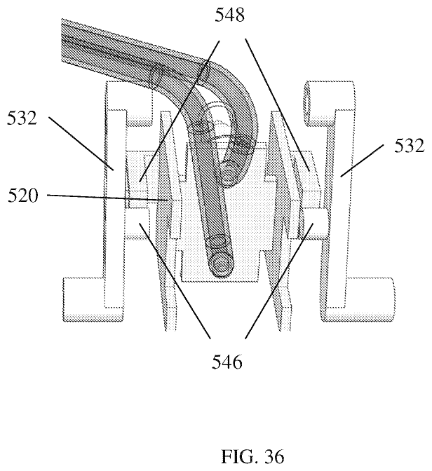

In some embodiments, as shown in FIG. 36, a boss 546 on an inner surface of the spike link 532 interacts with a fin 548 on the spike plate 520 to convert the rotation of the spike link 532 to a horizontal displacement of the spike plate 520. The linkage mechanism 530 may be provided on both sides of the pressure chamber in order to ensure smooth movement of the spikes 511, 512. The 4-bar linkage further helps to maintain the space envelope that already have for the pharmacy filled cassette such that a single design cassette may be used for both pharmacy filled and factory filled embodiments. The length of arms and other features reduce the spiking force as much as possible

In some embodiments, as shown in FIG. 37, the spike plate 520 may be guided by more than one bearing 522 on the valve block chassis 500. FIG. 37 shows a top perspective view of a portion of the valve block chassis in which the spike plate is disposed. The bearings 522 may engage top and bottom portions of opposing walls 562 of the spike plate. The bearings 522 aid in smooth linear movement of the spike plate when driving the spikes 511, 512. In some embodiments, tubing connected to ports 563, 564 may include slack to a connection of the drug paths to be maintained throughout the spike displacement.

FIGS. 38 and 39 show the spikes 511, 512 before and after piercing the septums 503, 504. After piercing, the spikes 511, 512 are disposed withing the bag ports 505, 506 of the drug delivery bags. The spikes have channels that extend a longitudinal length of each spike to permit drug to flow through the spikes. In some embodiments, the spike sheaths 514 move aside during piercing and drug can flow through the holes in the spikes. In some embodiments, the septum may an elastomeric septum that forms a seal around the spike after piercing.

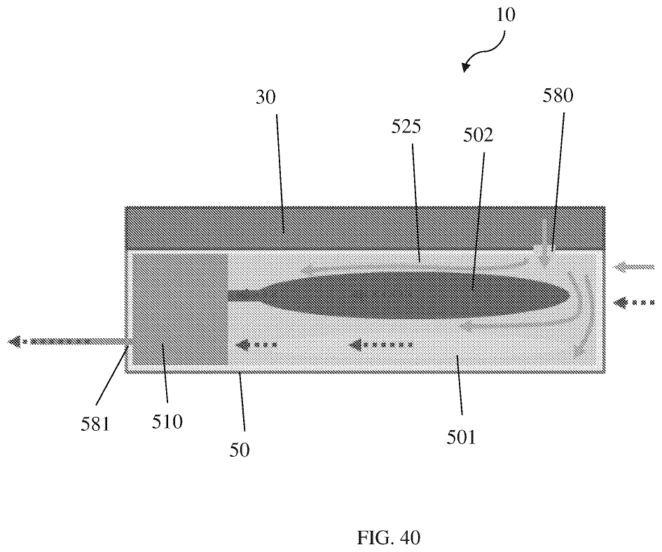

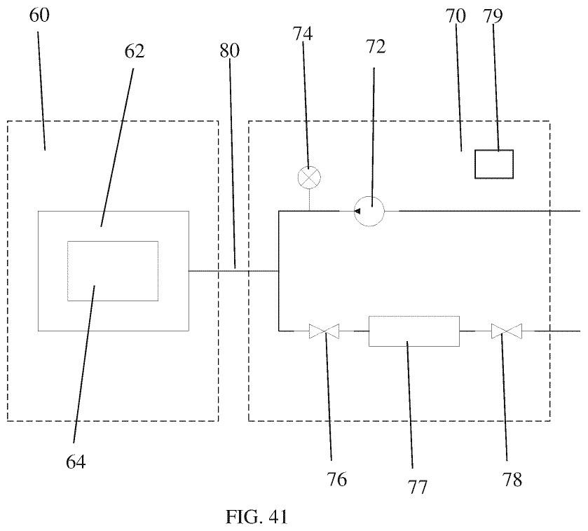

Once the septums are pierced and the drug flow path to the valve block assembly 510 has been opened, the cassette 50 and drug delivery system operate similarly to the embodiments described above. FIG. 40 is a schematic showing the operation of a drug delivery system using a pump module 30 and a cassette 50. It should be noted that the schematic applies to cassette 20 described in FIGS. 1-24. The pump module 30 pumps air into a pressure chamber 525 through a pneumatic interface 580 of the cassette 50 to pressurize the pressure chamber 525. The air applies pressure to drug delivery bags 501, 502 which squeezes the bags and causes a liquid drug to flow into a valve block assembly 510. The drugs follow the drug path through the valve block assembly to outlet port 581. A patient needle set attached to the outlet port 581 infuses the drugs into a patient subcutaneously.

A benefit to the drug delivery system is having only one pneumatic interface. One issue with shelf life is the transport of oxygen in or moisture out of the drug delivery bags. In the factory filled embodiment, once the drug delivery bags are filled and packaged within a cassette 50, the pneumatic interface may be sealed from the external environment. In some embodiments, inert gas may be added before sealing. Preventing oxygen transport may increase the shelf life of the cassette 50.

The drug delivery system described herein provides a simple drug infusion therapy of one or more drugs using a single-use cassette driven by a reusable pump. Because the cassette is driven by a reusable pump to deliver the drugs, minimal or no electronics or sensors are included in the cassette to maintain a compact, portable footprint. Drug infusions may take several hours, and patients may appreciate monitoring the status of the infusion therapy and thus it would be beneficial to estimate the volume of drug remaining in drug delivery bags throughout the infusion process. Accordingly, the drug delivery system provides a method for estimating the remaining drug volume without providing sensors in the cassette.

FIG. 41 is a schematic of a drug delivery system including a cassette 60 and a pump module 70. Although the schematic has been simplified to remove components that are not relevant to drug volume estimation, the cassette 60 and pump module 70 may be similar to and include features of other embodiments of cassettes and pump modules described herein. As shown in FIG. 41, in some embodiments, the cassette 60 includes a pressure chamber 62 and one or more drug delivery bags 64 disposed in the pressure chamber 62. In some embodiments, the cassette 60 is connected to the pump module 70 via a pneumatic interface 80.

In some embodiments, the pump module 70 includes a pump 72 and a pressure sensor 74 that is directly coupled to the pressure chamber 62 in the cassette 60. The pressure sensor is electronically interfaced with a device controller 79 in the pump module 70. In some embodiments, the system may include multiple pressure sensors that may operate across different pressure ranges to improve accuracy. The system may also include a pressure sensor for measuring atmospheric pressure. In some embodiments, the pump module 70 also includes a first valve 76, a metering chamber 77, and a second valve 78 arranged in series and coupled to the pressure chamber 62 in the cassette 60. In some embodiments, the first and second valves are electronically operable and may be electrically interfaced with the device controller 79. As shown the pressure chamber 62 is connected, via the first valve 76, to the metering chamber 77. In addition, the metering chamber 77 is connected, via the second valve 78, to atmosphere. In some embodiments, the metering chamber 77 has a smaller volume than the pressure chamber 62. The size of the metering chamber 77 maximized to provide more accuracy in the method, but small enough in size to fit within a portable drug delivery system. In some embodiments, the metering chamber 77 volume is approximately 60 mL. In some embodiments, the metering chamber may have a volume that ranges between 30 mL to 100 mL. In some embodiments, the pressure chamber volume may be approximately 300 mL to 1500 mL. In some embodiments, the ratio of a volume of the pressure chamber to a volume of the metering chamber may range between approximately 20:1 and 22:1. In one embodiment, the ratio may be approximately 21.6:1. In some embodiments, the ratio may range from 50:1 to 3:1.

In some embodiments, the volume of the drug delivery bag 64 may be estimated by calculating the difference between the total volume of the pressure chamber 62 (i.e., the volume with unfilled drug delivery bags, which is known) and the volume of air contained within the pressure chamber 62. The volume of air in the pressure chamber 62 will increase during the infusion process as the drug is dispensed from the drug delivery bag. Because there are no sensors in the cassette 60, the volume of air in the pressure chamber 62 cannot be directly measured. However, the volume of air in the pressure chamber 62 may be estimated using the algorithm described below.

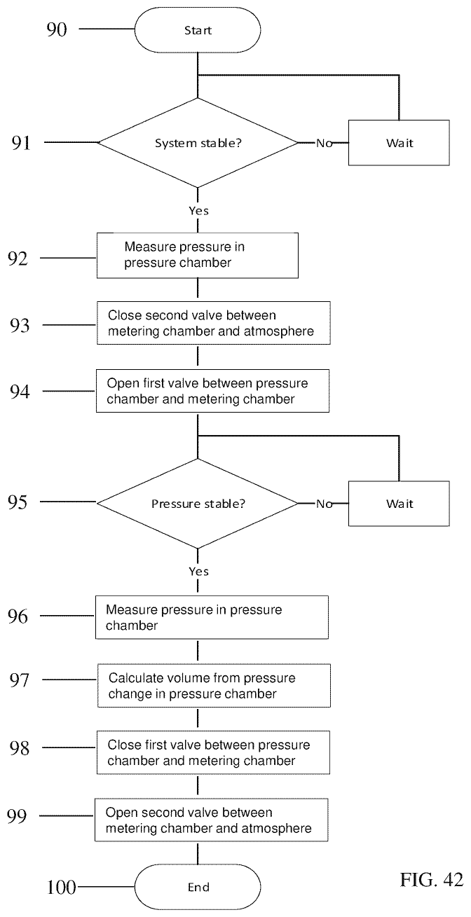

FIG. 42 is a flow chart of a method for estimating the volume of a drug delivery bag 64. The method provides a non-contact, pressure-based estimation of drug delivery bag volume without adding any components in the drug flow path or additional sensors to the single use cassette 60. At the start of the method (box 90), the first valve 76 is closed and the second valve 78 is open to allow the metering chamber 77 to equilibrate with atmosphere. An atmospheric pressure sensor, separate from the metering chamber, may measure the atmospheric pressure. Once the metering chamber is equilibrated with the atmosphere, the pressure of the metering chamber will be equal to the pressure measured by the atmospheric pressure sensor. The pump 72 may pressurize the pressure chamber 62 by pumping air through the pneumatic interface 80 into the pressure chamber. The pump may not run during the volume estimation method. The pressure chamber 62 is sealed.

At box 91, a check is made as to whether the system is stable. The method only proceeds to box 92 when the system stabilizes.

At box 92, once the system is stable, the pressure sensor 74 measures the air pressure in the pressure chamber and records a first pressure reading. As noted above, the pressure sensor 74 may include multiple pressure sensors that operate at different pressure ranges to improve accuracy.

At box 93, the second valve 78 between the metering chamber 77 and atmosphere is closed to seal the metering chamber 77 from atmosphere. Both the first valve 76 and the second valve 78 are now closed.

At box 94, the first valve between the pressure chamber 62 of the cassette 60 and the metering chamber 77 of the pump module 70 is opened. Due to the pressure differential between the pressure chamber 62 and the metering chamber 77, the air from the pressure chamber 62 undergoes an expansion to equalize the pressure across the pressure chamber, the metering chamber, and the intermediate pipework, which forms a connected volume. As a result, the pressure in the pressure chamber is reduced.

At box 95, again a check is made as to whether the system is stable. The method only proceeds to box 96 when the system stabilizes. The temperature of the air in the pressure chamber will reduce during the expansion process, and because air pressure is proportional to its temperature, temperature must be allowed to equilibrate before the second pressure sample is obtained to increase accuracy.

At box 96, once stable, the pressure sensor 74 measures the air pressure in the pressure chamber 62 and records a second pressure reading.

At box 97, the volume of air within the pressure chamber 62 is calculated using the following: the first pressure reading of the pressure chamber, the initial pressure in the metering chamber (e.g., equal to atmospheric pressure), the second pressure reading of the pressure chamber, the known volume of the metering chamber and its associated pneumatic tubing (e.g., connecting the pressure chamber to the metering chamber). Using these values, the volume of air inside the pressure chamber 62 may be calculated using the Ideal Gas Law. The volume occupied by the drug delivery bag 64 may then be estimated by subtracting the volume of air in the pressure chamber 62 from the total known volume of the pressure chamber 62 (which is known).

At box 98, the first valve 76 is closed to seal the pressure chamber 62.

At box 99, the second valve 78 is open to allow the metering chamber 77 to equilibrate with atmosphere and reset the system.

The method ends at step 100. After a delay to allow the pressure to stabilize, the method may start back at box 90. In some embodiments, volume estimations may be obtained at regular intervals throughout infusion process. It should be noted that since the volume estimation is carried out during the therapeutic drug delivery, outflow from the drug delivery bags will reduce the air pressure in the pressure chamber 62 more than expected from the expansion process alone. To increase accuracy, prior pressure gradient data may be used to apply a correction to the second pressure reading by subtracting the pressure gradient due to the drug flowing out of a drug delivery bag.

Graph 1: Pressure of Pressure Chamber

[symbol]

The pressure of the pressure chamber may look like Graph 1, shown above. The pressure (y axis) may decrease over time (x axis) as a drug flows out of the pressure chamber causing the air volume to increase in size, resulting in a decaying pressure. When the valve between the pressure chamber and the metering chamber is opened (e.g., metering event), there is a pressure drop (vertical line on graph), with the initial pressure chamber gradient on the left and the final pressure chamber pressure gradient on the right. The final pressure reading is ideally taken at time zero of the metering event, but there may be transient effects like temperature and noisy pressure sensors, so pressure readings may be taken over time. A correction may be applied to estimate this final pressure of the pressure chamber after the metering event as shown in Graph 2, below. The vertical dashed line represents the volume estimation event, the top horizontal line represents the initial pressure chamber pressure, and the bottom horizontal line represents the final pressure chamber pressure.

Graph 1: Pressure Gradient Correction

[symbol]

In addition to estimating the volume of a drug delivery bag, it may be useful to estimate a drug flow rate during the drug delivery process. An estimated drug flow above or below a predetermined rate may be used to detect fault conditions such as a disconnected needle set, pinched tube, or outflow occlusion.

Returning to FIG. 41, the components used in the flow detection method include the pressure chamber 62 and drug delivery bags 64 of the cassette 60 and the pump 72 and the pressure sensor 74 in the pump module 70. A pneumatic interface 80 connects the pump module 70 to the cassette 60. The pressure sensor 74 is coupled directly to the pressure chamber 62. The pressure sensor 74 is electronically interfaced with a device controller 79. The system also includes an ambient temperature sensor (not shown) electronically interfaced with the device controller 79 to measure the ambient temperature of the pressure chamber to determine a temperature of a drug in a drug delivery bag. In some embodiments, if the temperature is below an acceptable temperature, the system may alert the user to warm up the drug delivery bag before initiating the infusion process. Running within the software of the device controller 79 is a computational algorithm responsible for converting inputs from the pressure sensor 74 and ambient temperature sensor into a flow rate estimate.

The pressure sensor 74 monitors pressure decay inside of the pressure chamber 62. To dispense the therapeutic drug from the drug delivery bag 64, the pressure chamber 62 is pressurized and then sealed, which pneumatically compresses the drug delivery bag 64. It should be noted that with both the volume estimation and flow detection methods, one or more drug delivery bags may be disposed in the pressure chamber 62.

As the therapeutic drug is dispensed, the air pressure in the pressure chamber 62 decays and the surrounding air expands. When the pump is off the air pressure in the pressure chamber will reduce as it expands to fill a larger volume (e.g., as the drug is dispensed). When the pump is on, however, the air pressure will not reduce. A rate of pressure decay may be calculated by capturing a series of pressure readings of the pressure chamber at known time intervals. The air volume of the pressure chamber may be calculated using the methods described above. The pressure decay rate and the air volume may then be used to calculate an estimate for a drug flow rate. This estimate drug flow rate may be compared to a target or expected flow rate to detect an out of bound event and trigger an alarm. Accordingly, an indirect and non-contacting method of estimating the rate of fluid outflow may be provided.

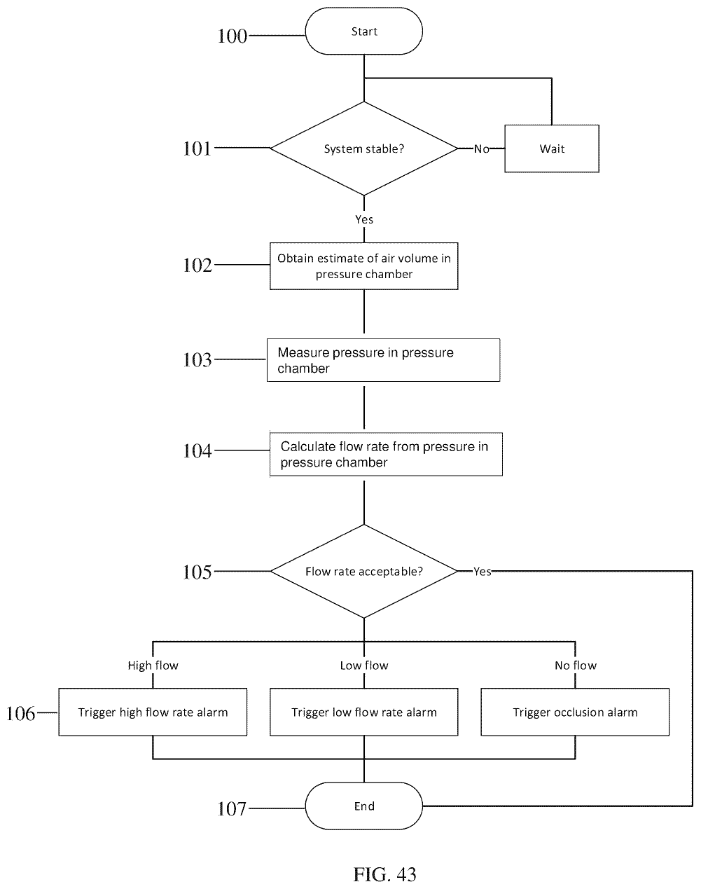

FIG. 43 is a flow chart of a flow detection method. The flow detection method is conducted during a therapeutic delivery of a specific drug using a specific needle set. Prior to the start of the method (step 100), the following information, which is specific to the therapeutic drug being delivered and needle set used to deliver the drug (e.g., type of needle and number of infusion sites), must be collected: the relationship between the slope of pneumatic pressure decay and drug delivery rate, from a maximum flow rate to no-flow, across a range of recommended operating temperatures. Based on this information, a predetermined target flow rate may be calculated based on the specific therapeutic drug being delivered using the specific needle set. A temperature sensor may measure the temperature of the drug in the drug delivery bag to determine the required drive pressure in the first chamber to achieve a desired drug flow rate.

At box 101, a check is made as to whether the system is stable. The method only proceeds to box 102 when the system stabilizes.

At box 102, once the system is stable, an estimate of the current volume of air within the pressure chamber 62 is obtained. The method for estimating the volume of air in the pressure chamber 62 is described above with respect to FIG. 42 regarding volume estimation. This estimate may be used to estimate the volume of drug in a drug delivery bag.

At box 103, at prescribed intervals, the pressure input to the pressure chamber is sealed off and the pressure is sampled at a prescribed rate until a target number of samples has been collected. From the collected data, the rate of pressure decay may be calculated.

At box 104, the drug flow rate is calculated based on the volume of air in the pressure chamber and the rate of pressure decay. The measured slope of the pressure decay is compared against reference data to calculate the drug flow rate. The calculated drug flow rate is compared to a predetermined target flow rate based on reference data specific to the type of drug and needle set combination being used for the drug delivery therapy. For example, a pressure decay slope which is steeper than expected indicates a leak, while a shallower slope indicates reduced flow (for instance due to a pinched tube), and the absence of a slope indicates occlusion.

At box 105, the system checks whether the calculated flow rate is acceptable. In some embodiments, the estimated flow rate may be considered acceptable if it is equal to or within plus or minus 10% of the expected flow rate. If the system determines that the estimated drug flow rate is an acceptable flow rate, then the method skips to box 107 and terminates.

If the system estimates an abnormal flow rate (e.g., high, low, or no flow), then the method proceeds to box 106 and triggers an alarm in the form of a visual or audio warning to alert a user of a potential issue. For example, a pressure decay slope that is steeper than expected (e.g., high flow rate) may indicate a possible leak. A shallower slope (e.g., low flow rate) may indicate a pinched tube, and the absence of a slope (e.g., no flow) may indicate occlusion.

In some embodiments, the method may include a correction to the estimated flow rate to account for temperature. The temperature may affect the viscosity of a drug in a drug delivery bag which may affect the drug flow rate at a fixed pressure. For example, a drug may have a higher viscosity at colder temperatures which may lead to a slower flow rate. In some embodiments, a correction may be applied to the measured flow rate to account for the temperature and to minimize any temperature effect on the measured flow rate. In the event a flow rate is measured below the acceptable flow rate, the correction can help ensure that the slower flow rate is due to an occlusion or other issue in the drug flow path and not due to temperature.

In both the volume estimate and flow detection methods, the system may send data directly to an app for the user to view and/or manage.

According to one aspect, a pump module may be compatible with cassettes of different sizes. Cassette sizes may vary in order to hold different volumes of drug. In some embodiments, cassettes of different sizes may have congruent pump module interfacing areas so that the same pump module may be used with each of these different cassette sizes.

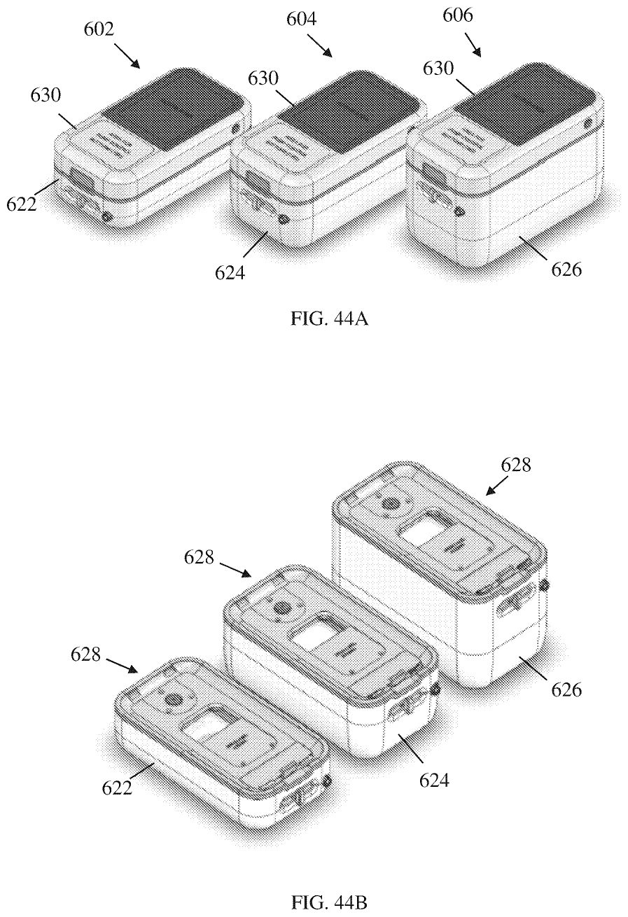

In one illustrative embodiment shown in FIGS. 44A and 44B, a plurality of drug delivery systems 602, 604, 606 utilizing three different cassettes of different sizes is provided. The first cassette 622 has the smallest size, the second cassette 624 has a medium size, and the third cassette 626 has the largest size. FIG. 44A shows each of the different cassettes interfacing with a pump module 630. FIG. 44B shows the cassettes with the pump modules removed. In this illustrative embodiment, each of the cassettes has a pump module interfacing area 628 that is congruent across the three different cassette sizes. In some embodiments, to accommodate different volumes of drug, the height of the cassettes may be varied, with the tallest cassette 626 being able to hold the largest amount of drug, and the shortest cassette 622 being able to hold the smallest amount of drug. Alternatively or in addition, in some embodiments, the width and/or length of the cassette may be varied to accommodate different volumes of drug. While three different cassette sizes are shown in FIGS. 44A and 44B, it should be appreciated that any suitable number of sizes may be provided.

As discussed above, the cassettes may contain one or more drug delivery bags (or “drug bags”) in which drug, such as HyQvia, is held. According to one aspect, a cassette may include trays to support the drug bags.

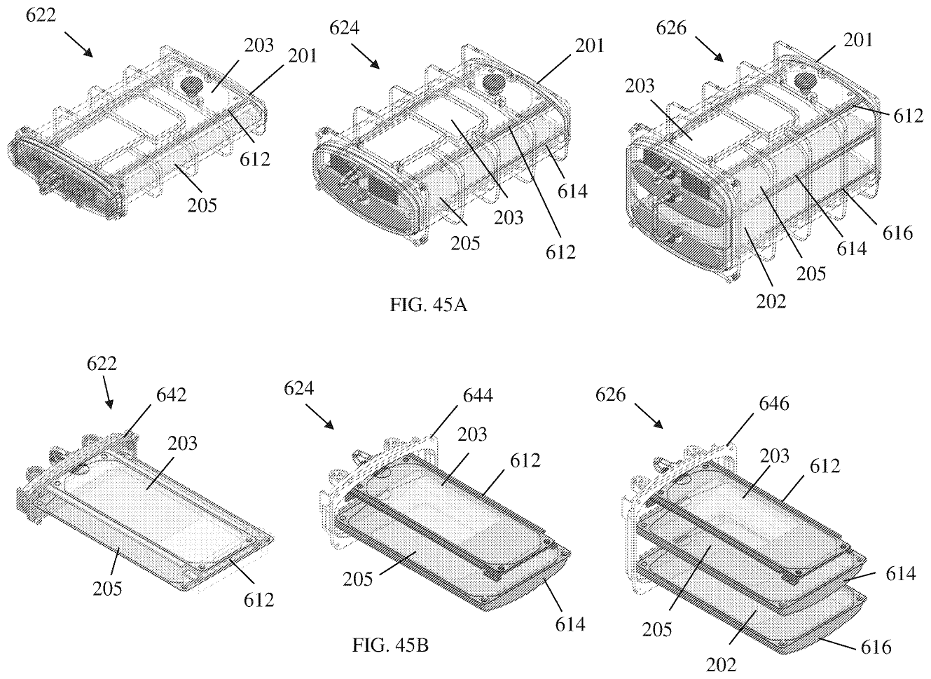

In the illustrative embodiment shown in FIG. 45A, the pressure chambers 201 of each of the cassettes 622, 624, 626 are shown in phantom to reveal the trays and drug delivery bags within. In FIG. 45B, the pressure chambers are hidden altogether. As seen in FIGS. 45A and 45B, the first cassette 622, which is the smallest cassette, may have only a single tray, a first tray 612. This first tray 612 accommodates two drug delivery bags, 203 and 205. The second cassette 624, which is a medium-sized cassette, may have two trays: a first tray 612 that supports a first drug delivery bag 203, and a second tray 614 that supports a second drug delivery bag 205. Finally, the third cassette 626, which is the largest cassette, may have three trays: a first tray 612 that supports a first drug delivery bag 203, a second tray 614 that supports a second drug delivery bag 205, and a third tray 616 that supports a third drug delivery bag 202. It should be appreciated that any number of trays and drug delivery bags may be used.

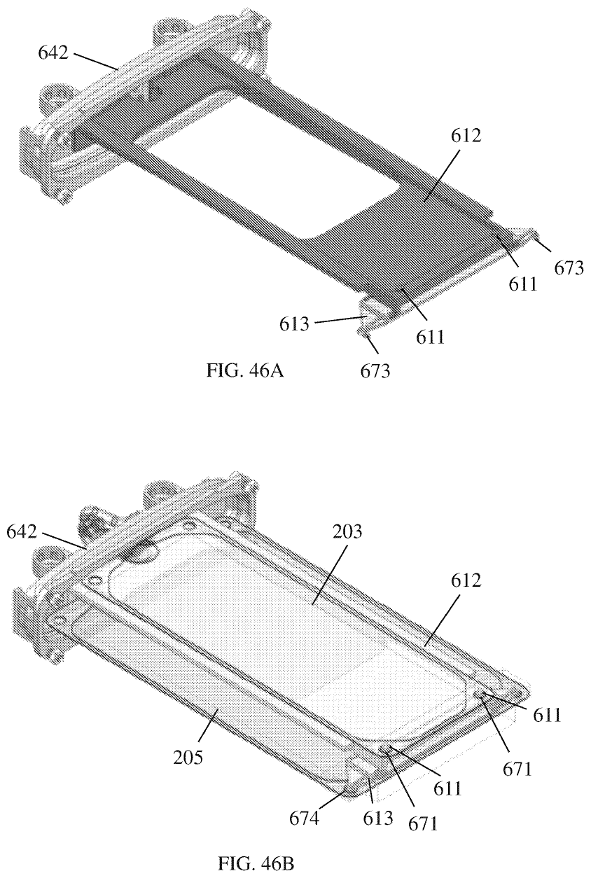

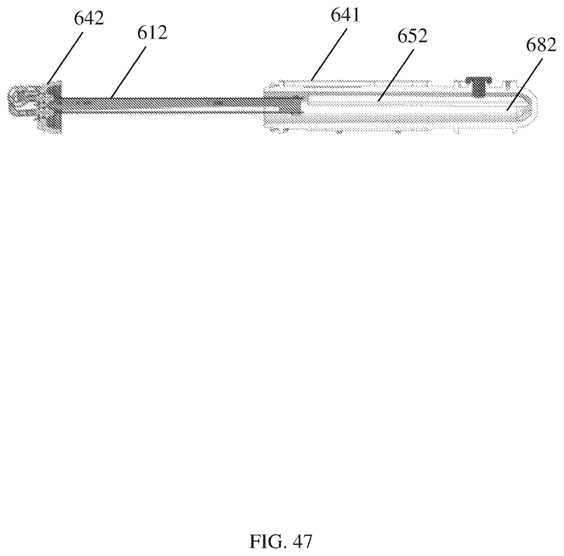

FIGS. 46A, 46B and 47 show the tray arrangement of the first cassette in more detail. As shown in FIG. 46A, the first cassette has a first tray 612. In some embodiments, the tray 612 may include posts 611 that are sized and positioned to be received by openings 671 in the first drug delivery bag 203. The posts may serve to loosely hold the bags in position on the tray. In the illustrative embodiment shown in FIGS. 46A and 46B, the cassette has only a single tray. In some embodiments, even with the single tray, the cassette may still hold two drug delivery bags. In some embodiments, a bag clip 613 may attach to the first tray 612, and the bag clip 613 may serve to hold the second drug delivery bag 205 in position. The bag clip 613 may have posts 673 that are sized and positioned to be received by openings 674 in the second drug delivery bag 205.

As shown in the cross-sectional side view of FIG. 47, the pressure chamber may include a pressure chamber body 641 and a pressure chamber cap 642. One end of the tray 612 may be attached to the pressure chamber cap 642 such that the tray 612 may move with the pressure chamber cap 642. The pressure chamber body 641 may include a tray rib 652 on the sidewall 682 (a second tray rib may mirror the rib 652 on an opposing sidewall such that the tray 612 has a corresponding pair of tray ribs). The tray 612 may be configured to slide onto the tray ribs 652 when the pressure chamber cap 642 is closed onto the pressure chamber body 641. The tray ribs 652 may serve to support the tray 612 within the pressure chamber.

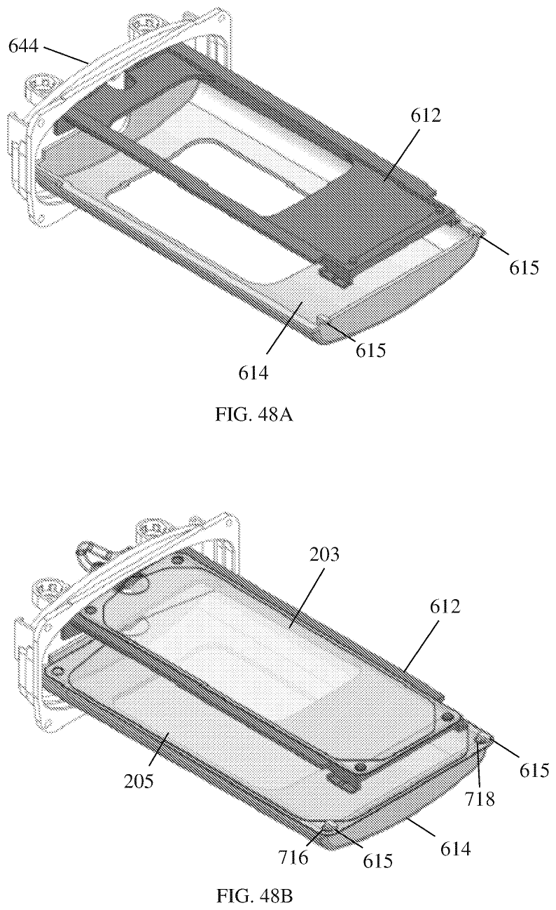

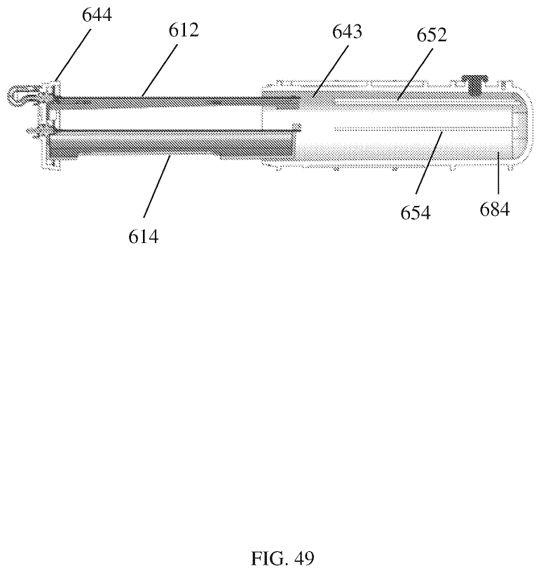

FIGS. 48A, 48B and 49 show the tray arrangement of the second cassette in more detail. As shown in FIG. 48A, the second cassette has a first tray 612 and a second tray 614, each holding its own drug delivery bag. The first tray 612 holds a first drug delivery bag 203, and the second tray 614 holds a second drug delivery bag 205. The first tray 612 may be structurally similar to that of the first cassette in FIGS. 46A, 46B, and 47. Instead of a bag clip to hold a second drug delivery bag, however, the second cassette has a designated tray, the second tray 614, to hold the second drug delivery bag 205.

As shown in the cross-sectional side view of FIG. 49, the pressure chamber may include a pressure chamber body 643 and a pressure chamber cap 644. One end of the first tray 612 and the second tray 614 may be attached to the pressure chamber cap 644 such that the trays 612, 614 may move with the pressure chamber cap 644. The pressure chamber body 643 may include a first tray rib 652 and a second tray rib 654 on the sidewall 684 (additional ribs may mirror the ribs 652, 654 on an opposing sidewall such that each tray has a corresponding pair of tray ribs). The first tray 612 may be configured to slide onto the first pair of tray ribs 652, and the second tray 614 may be configured to slide onto the second pair of tray ribs 654 when the pressure chamber cap 644 is closed onto the pressure chamber body 643. The first pair of tray ribs 652 may serve to support the first tray 612 within the pressure chamber, and the second pair of tray ribs 654 may serve to support the second tray 614 within the pressure chamber.

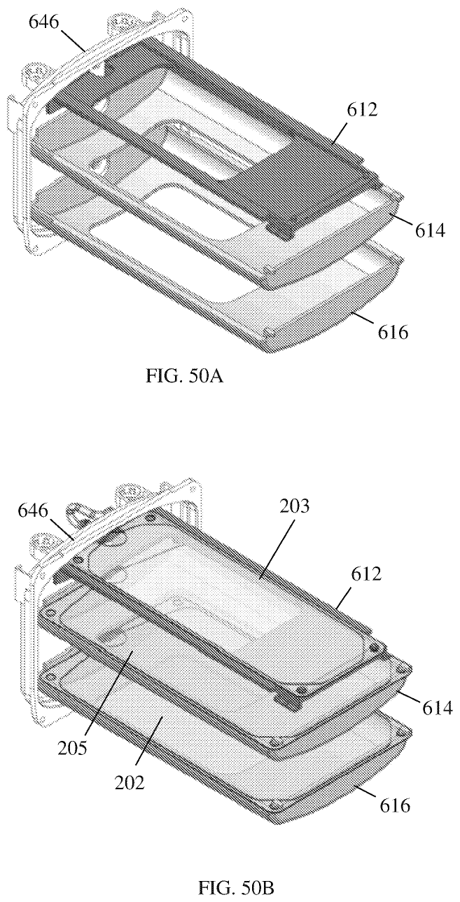

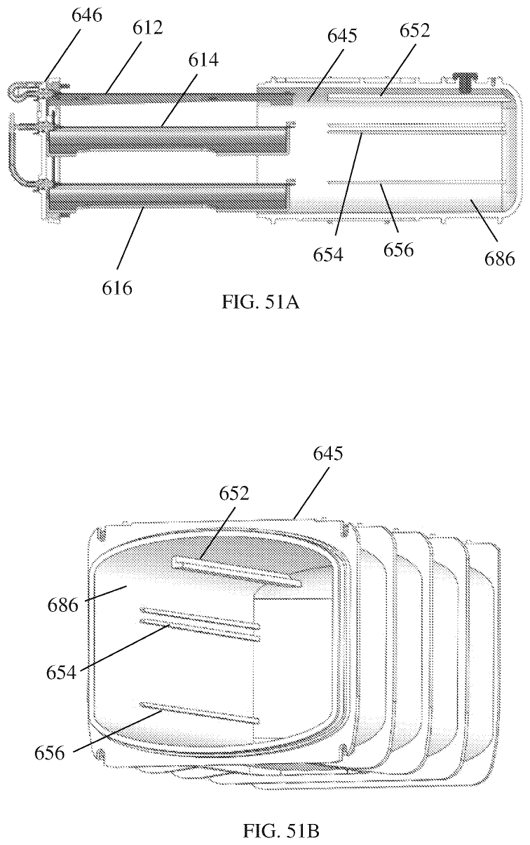

FIGS. 50A, 50B, 51A and 51B show the tray arrangement of the third cassette in more detail. As shown in FIG. 50A, the second cassette has a first tray 612, a second tray 614, and a third tray 616, each holding its own drug delivery bag. The first tray 612 holds a first drug delivery bag 203, the second tray 614 holds a second drug delivery bag 205, and the third tray 616 holds a third drug delivery bag 202. The first tray 612 may be structurally similar to that of the first cassette in FIGS. 46A, 46B, and 47. Instead of a bag clip to hold a second drug delivery bag, however, the second cassette has a designated tray, the second tray 614, to hold the second drug delivery bag 205. The second tray 614 and third tray 616 may be structurally similar to that of the second cassette in FIGS. 48A, 48B, and 49.

As shown in the cross-sectional side view of FIG. 51A, the pressure chamber may include a pressure chamber body 645 and a pressure chamber cap 646. One end of the first tray 612, the second tray 614, and the third tray 616 may be attached to the pressure chamber cap 646 such that the trays 612, 614, 616 may move with the pressure chamber cap 646. The pressure chamber body 645 may include a first tray rib 652, a second tray rib 654, and a third tray rib 668 on the internal sidewall 686 (additional ribs may mirror the ribs 652, 654, 656 on an opposing sidewall such that each tray has a corresponding pair of tray ribs). The first tray 612 may be configured to slide onto the first pair of tray ribs 652, the second tray 614 may be configured to slide onto the second pair of tray ribs 654, and the third tray 616 may be configured to slide onto the third pair of tray ribs 656 when the pressure chamber cap 646 is closed onto the pressure chamber body 645. The first pair of tray ribs 652 may serve to support the first tray 612 within the pressure chamber, the second pair of tray ribs 654 may serve to support the second tray 614 within the pressure chamber, and the third pair of tray ribs 656 may serve to support the third tray 616 within the pressure chamber. A perspective view of the pressure chamber body 645 showing the tray ribs 652, 654, 656 is shown in FIG. 51B.

For each of the first, second, and third cassettes, in some embodiments, the type of drug contained in the first drug delivery bag is different from the type of drug contained in the second drug delivery bag. In some embodiments, for the third cassette, the type of drug contained in the second drug delivery bag may be the same as the type of drug contained in the third drug delivery bag.

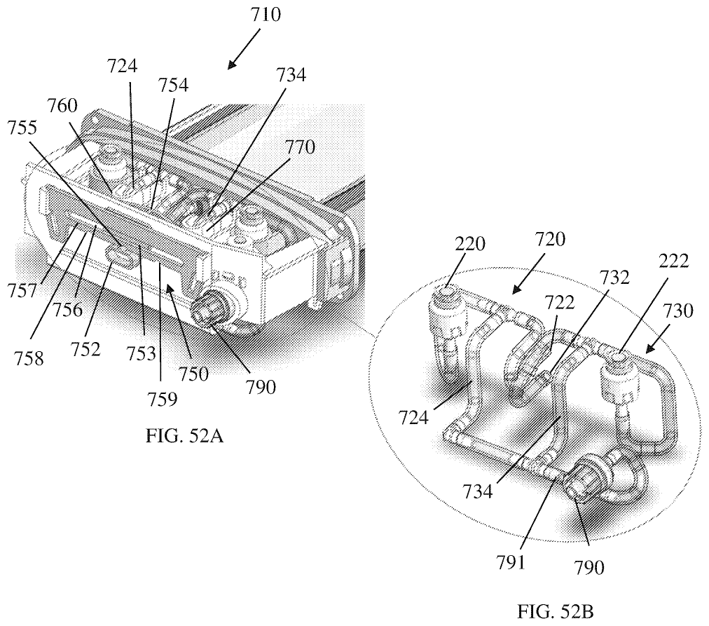

As discussed above, a drug delivery system may include a valve block assembly configured to control drug flow from one or more drug delivery bags. An alternative embodiment is shown in FIGS. 52A-55B, where the valve block assembly utilizes pinch valves to control drug flow. Components with the same numerical labels as those used in earlier figures are similar or identical to the previously embodiments.

FIG. 52A shows a perspective view of the valve block assembly 710 having a valve switch 750. The valve switch 750 may be moveable by a user to control drug flow from one or more drug delivery bags. The valve switch 750 may include a selector 752 that a user may slide linearly between different options. The valve switch 750 may be fixed to a contact ramp 754 and a tab 753. The tab 753 may slide within a slot 757 of a backplate 756 of the valve switch 750. Thus, as the user slides the valve switch 750 to different positions, the contact ramp 754 also moves to different positions.

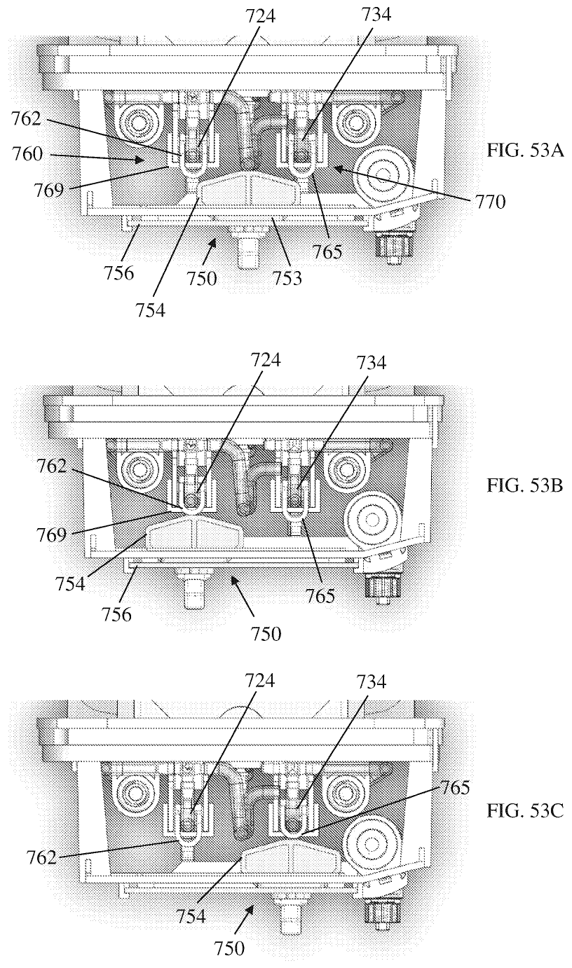

The contact ramp 754 interacts with a first pinch valve 760 and a second pinch valve 770 to control drug flow. In the illustrative embodiment of FIGS. 52A-53C, the first and second pinch valves are normally closed valves. Each pinch valve is configured to open by way of contact of the contact ramp 754 against the valve.

In the illustrative embodiment of FIGS. 52A-53C, the contact ramp 754 has three positions: a first position in the center (as seen in FIG. 52A and FIG. 53A) where the contact ramp 754 is out of contact with both pinch valves 760, 770; a second position (as seen in FIG. 53B) where the contact ramp 754 is in contact with the first pinch valve 760; and a third position (as seen in FIG. 53C) where the contact ramp 754 is in contact with the second pinch valve 770. In some embodiments, the valve switch may include detents for each of these positions of the contact ramp 754. For example, as shown in FIG. 52A, the backplate 756 may have detents 758, 759 that interact with a notch 755 on the selector 752. The detents may be configured to maintain the valve switch in a desired blocking position.

When the valve switch 750 is in the second position, shown in FIG. 53B, the contact ramp 754 abuts against a contact 762 of the first pinch valve 760, causing the pinch valve 760 to open, and thereby permitting drug to flow through a first tubing 724 of a first drug path 720. When the valve switch is 750 is in the third position, shown in FIG. 53C, the contact ramp 754 abuts against a contact 765 of the second pinch valve 770, causing the pinch valve 770 to open, and thereby permitting drug to flow through a second tubing 734 of a second drug path 730.

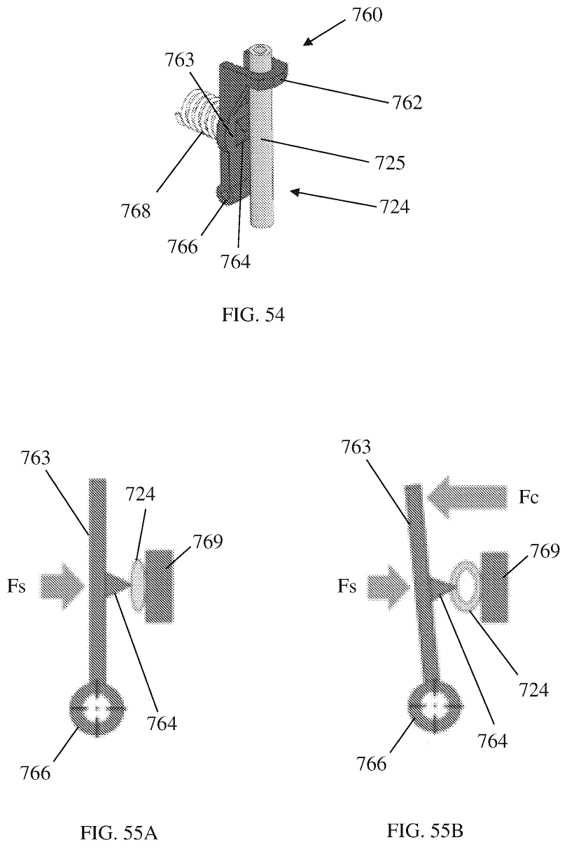

One illustrative embodiment of pinch valve 760 is shown in FIG. 54, with schematics of the mechanism of action shown in FIGS. 55A and 55B. The second pinch valve 770 may have similar structure. The pinch valve 760 may be biased by a spring 768 into a closed position, and thus may have a normally closed configuration when out of contact with the valve switch 750. The pinch valve may include a plate 763 with a protrusion 764 configured to contact and pinch a pinch region 725 of the tubing 724 closed. The plate 763 may be configured to pivot about a hinge 766.

In FIG. 55A, the pinch valve is shown in the normally closed configuration. The spring 768 exerts a spring force Fs on the plate 763, urging the protrusion 764 against the tubing 724. The tubing 724 is pinched between the protrusion 764 and a contact surface 769 of the pinch valve, causing the tubing 724 to be pinched closed. In FIG. 55B, the contact ramp 754 has been moved into contact with the pinch valve, exerting a contact force Fc that overcomes the spring force Fs, causing the plate 763 to rotate about hinge 766 away from the tubing 724, thus allow the tubing 724 to open. Drug can now flow through the tubing 724 and out of an outlet port 790 (see FIG. 52B).

As seen in FIG. 52B, the valve block includes two drug flow paths: first drug path 720 and second drug path 730. The first tubing 724 serves as the section of the first drug path 720 that is pinched by the first pinch valve 760. The second tubing 734 serves as the section of the second drug path 730 that is pinched by the second pinch valve 770. In some embodiments, the portions of the drug paths that are pinched may be made of a different material than tubing of other parts of the drug path. For example, the pinched tubing sections may be made of materials that are more flexible than tubing of other parts of the drug path to facilitate pinching.

As discussed above, in some embodiments, the pump module and cassette may include a sensing arrangement to detect the position of the valve switch of the cassette. The sensing arrangement may allow a controller of the pump module to determine the state of the switch. In some embodiments, optical detection may be used in the sensing arrangement.

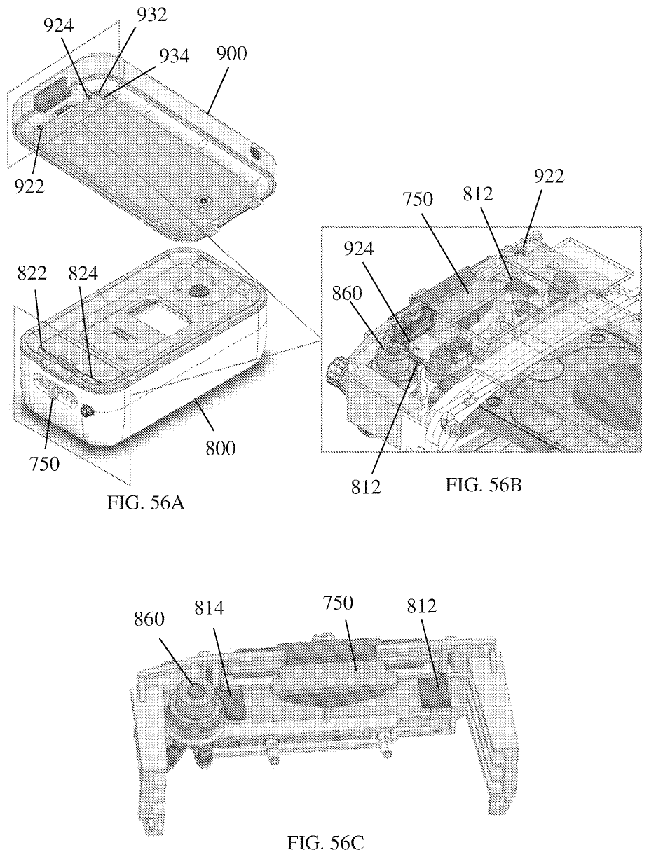

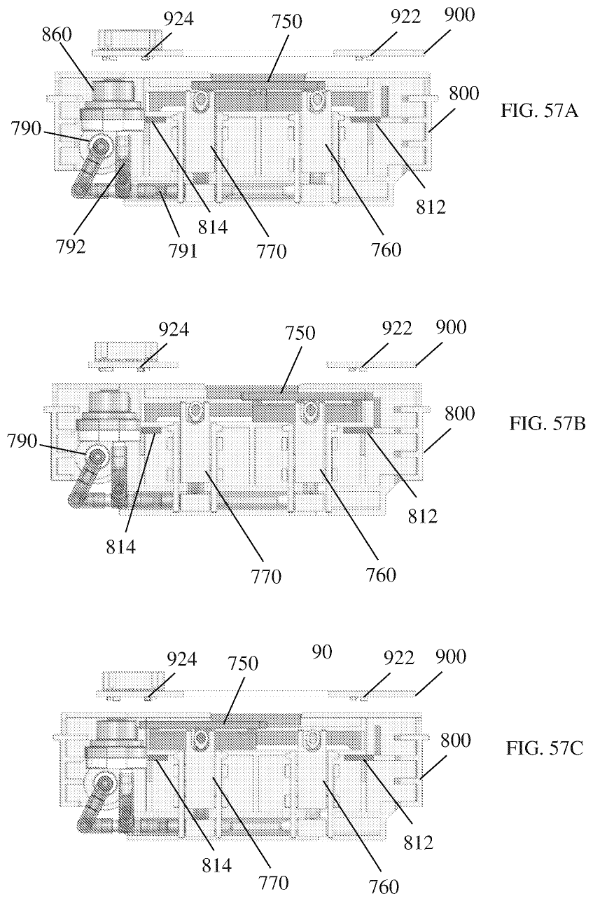

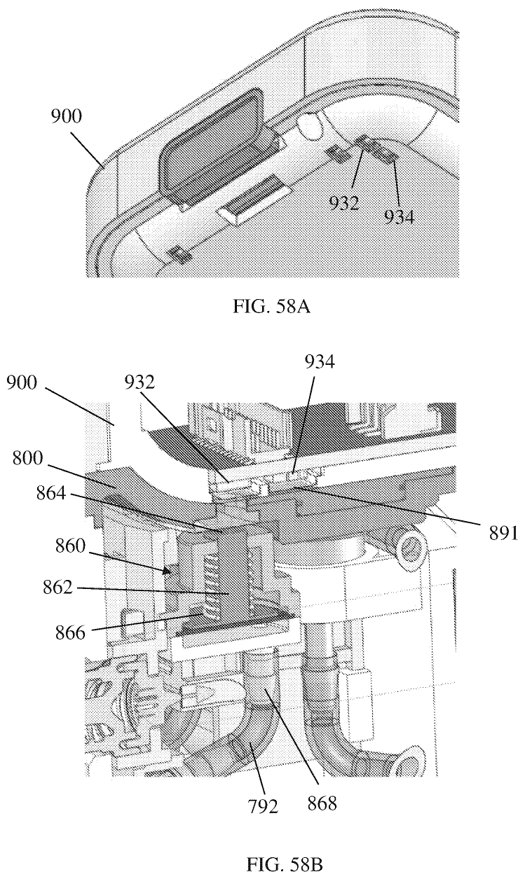

One illustrative embodiment of a valve switch sensing arrangement is shown in FIGS. 56A-57C. The sensing arrangement is configured to determine the position of the valve switch 750. This may allow the pump module to determine which drug has been selected for administration (or if no drug is being administered). The pump module 900 includes a first optical sensor 922 and a second optical sensor 924 that align with optical windows 822, 824 respectively on the cassette 800. As the valve switch 750 is moved between positions, at least a portion of the valve switch 750 may appear through the optical windows 822, 824.

In some embodiments, a valve switch may have three positions: a first position in which no drug is selected (see FIG. 57A and FIG. 53A), a second position in which a first drug is selected (see FIG. 57B and FIG. 53B), and a third position in which a second drug is selected (see FIG. 57C and FIG. 53C). A first reference surface 812 may be aligned with the first optical sensor 922, and a second reference surface 814 may be aligned with the second optical sensor 924.

As shown in FIG. 57A, when the valve switch is in the first position in which no drug is selected, both reference surfaces 812, 814 may be unobstructed by the valve switch 750 such that the optical sensors 922, 924 detect the reference surfaces 812, 814, respectively (e.g. there is a clear line of sight from the sensors 922, 924 to the reference surfaces 812, 814, respectively). The sensors send signals to the pump module controller indicating that both reference surfaces are detected, and from that information, the controller is able to determine that no drug has been selected.

As shown in FIG. 57B, when the valve switch 750 is in the second position in which the first drug is selected, the first reference surface 812 may become obstructed by the valve switch 750, where at least a portion of the valve switch 750 is positioned between the first reference surface 812 and the first optical sensor 922. The first sensor 922 sends a signal to the pump module controller indicating that the valve switch 750 is detected (and/or that the first reference surface 812 is not detected). From this information, the controller is then able to determine that the first drug has been selected. In some embodiments, the second sensor 924 may also send a signal to the pump module controller indicating that the second reference surface 814 is detected. The controller may, in some embodiments, determine that the first drug has been selected only when both the valve switch 750 is detected by the first sensor 922, and the second reference surface 814 is detected by the second sensor 924. However, in other embodiments, the first sensor 922 sensing the valve switch 750 is sufficient information for the controller to determine that the first drug has been selected.

As shown in FIG. 57C, when the valve switch 750 is in the third position in which the second drug is selected, the second reference surface 814 may become obstructed by the valve switch 750, where at least a portion of the valve switch 750 is positioned between the second reference surface 814 and the second optical sensor 924. With the valve switch 750 obstructing the second reference surface 814, the second optical sensor 924 no longer detects the second reference surface 814, and instead detects the presence of the valve switch 750. The second sensor 924 sends a signal to the pump module controller indicating that the valve switch 750 is detected (and/or that the second reference surface 814 is not detected). From this information, the controller is then able to determine that the first drug has been selected. In some embodiments, the first sensor 922 may also send a signal to the pump module controller indicating that the first reference surface 812 is detected. The controller may, in some embodiments, determine that the second drug has been selected only when both the valve switch 750 is detected by the second sensor 924, and the first reference surface 812 is detected by the first sensor 922. However, in other embodiments, the second sensor 924 sensing the valve switch 750 is sufficient information for the controller to determine that the second drug has been selected.

As discussed above, in some embodiments, the pump module and cassette may include a sensing arrangement to detect end of flow. The end of flow detector may indicate when a first drug delivery bag has been emptied to signal to the user to switch the valve block to open a subsequent drug path flow, and/or may indicate to the user the emptying of the last drug delivery bag for the given treatment sessions and that the drug therapy has completed. The sensing arrangement may allow a controller of the pump module to determine the state of flow. In some embodiments, optical detection may be used in the sensing arrangement.

One illustrative embodiment of an end of flow sensing arrangement is shown in FIGS. 56A-57C. The sensing arrangement is configured to determine the state of flow from one or more drug delivery bags. The pump module 900 includes a flow measurement sensor 932 and a reference sensor 934. As seen in FIG. 58B, when the pump module 900 and the cassette 800 are mated together, the flow measurement sensor 932 of the pump module 900 aligns with a piston surface 864 of the cassette 800, and the flow reference sensor 934 of the pump module 900 aligns with a reference surface 891 of the cassette 800.

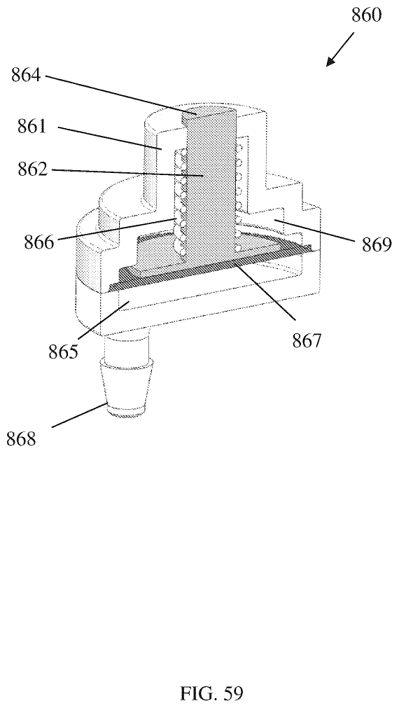

As seen in FIGS. 58B and 59, the piston surface 864 is part of a piston 862 of a flow detector 860. The flow detector 860 may operate in a similar manner to the flow detector 227 of FIGS. 21 and 22, except that the position of the piston may be detected using an optical sensor rather than a Hall-effect sensor.

The piston 862 is moveable relative to a housing 861 of the flow detector 860. The flow detector may have a first chamber 865 and a second chamber 869, and a diaphragm seal 867 that separates the first and second chambers. The piston 862 may be attached to the diaphragm seal 867, which may allow the piston to linearly move relative to the housing 861 while maintaining a seal between the first and second chambers 865, 869. The first chamber 865 includes an inlet 868 that may be fluidly connected to tubing 792 that is in fluid communication with the drug path(s).

As shown in FIG. 52B, a valve block assembly may include a first drug path 720 and a second drug path 730. Both drug paths may flow into an outlet path 791, which directs drug out through an outlet port 790. As shown in FIG. 57A, the tubing 792 may branch off the outlet path 791. As such, the fluid pressure in the tubing 792 may reflect the fluid pressure in the outlet path 791. The diaphragm seal 867 may serve to seal off fluid communication between the piston 862 and the tubing 792, as well as the outlet path 791.

The position of the piston surface 864 may change depending on whether there is drug flow in the tubing 792 (and the outlet path 791). When there is drug flow, the flow results in fluid pressure within the tubing 792 and the first chamber 865, which may act on the diaphragm 867, which in turn moves the piston 862 away from the first chamber 865 and towards the flow measurement sensor 932. When there is no drug flow in the tubing 792 and the outlet path 791, the force from the spring 866 biases the piston to move back toward the first chamber 865, and away from the flow measurement sensor 932. The flow measurement sensor 932 may be an optical sensor that is configured to determine this change in position of the piston surface 864, and may send a signal to a controller indicating that the position of the piston surface 864 has changed (e.g. has moved further away from the sensor 932). From this information, the controller may then determine that flow has ended and/or is soon to end.

In some embodiments, a references sensor 934 and a reference surface 891 may be used to assist in the end of flow detection. The reference sensor 934 and the reference surface 891 may be used to measure the distance of the pump module 900 from the cassette 800. This measured distance provides a reference measurement for the flow measurement sensor 932.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone, tablet, or any other suitable portable or fixed electronic device.

Also, a computing device may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A method for estimating a volume of a drug in a collapsible drug delivery bag, the method comprising:
measuring a first air pressure in the first chamber when a collapsible drug-filled drug delivery bag is disposed in the first chamber;
pneumatically connecting the first chamber to a second chamber;
measuring a second air pressure in the first chamber;
calculating a volume of air in the first chamber based on the first and second air pressure readings, an initial air pressure in the second chamber, and a known volume of the second chamber; and
subtracting the volume of air in the first chamber from a total volume of the first chamber.

2. The method of claim 1, further comprising the initial air pressure in the second chamber with atmospheric pressure prior to pneumatically connecting the first chamber to the second chamber.

3. The method of claim 2, further comprising sealing the second chamber by closing a second valve arranged between the second chamber and atmosphere prior to pneumatically connecting the first chamber to the second chamber.

4. The method of claim 1, wherein pneumatically connecting the first chamber to a second chamber comprises opening a first valve between the first chamber and a second chamber.

5. The method of claim 1, further comprising decreasing air pressure in the first chamber.

6. The method of claim 1, wherein calculating a volume of air in the first chamber comprising using the Ideal Gas Law.
